(19) United States
(12) Patent Application Publication
(10) Pub. No.: US 2025/0208103 A1
(43) Pub. Date: Jun. 26, 2025

(54) COMPREHENSIVE ANALYTICAL CHARACTERIZATION OF mRNA UNDER ONE OR MORE STRESS CONDITIONS

(71) Applicant: Regeneron Pharmaceuticals, Inc., Tarrytown, NY (US)

(72) Inventors: Jaclyn Cika, Suffern, NY (US); Yue Fu, Pleasantville, NY (US); Kathir Muthusamy, Cheshire, CT (US); Deanna Di Grandi, Goshen, NY (US); Nisha Palackal, White Plains, NY (US)

(21) Appl. No.: 18/986,093

(22) Filed: Dec. 18, 2024

Related U.S. Application Data
(60) Provisional application No. 63/612,597, filed on Dec. 20, 2023.

(51) Int. Cl. G01N 30/88; B01D 15/34; B01D 15/38; C12Q 1/48; G01N 30/02; G01N 30/74

(52) U.S. Cl. CPC G01N 30/88; B01D 15/34; B01D 15/3804; C12Q 1/485; G01N 30/74; C12Y 301/15; C12Y 306/01; G01N 2030/027; G01N 2030/8827

(57) ABSTRACT

The present inventions provide a concise panel of chromatography-based stability-indicating methods for evaluating in vitro transcribed (IVT) mRNA under various conditions, including varying types and degrees of stress conditions, as part of a forced degradation study. The inventions also provide that addition of EDTA to the mRNAs prior to heat exposure reduces the extent of mRNA degradation, that the transcripts are fragmenting via a divalent metal-ion mediated pathway. The inventions also provide the application of the methods to evaluate the critical quality attributes (CQAs) of mRNAs as well as to detect intrinsic process and product related impurities.

This application claims priority to U.S. Application Ser. No. 63/612,597, filed Dec. 20, 2023, which is incorporated by reference in its entirety.

### FIELD OF THE INVENTIONS

The present inventions relate to methods for evaluating in vitro transcribed (IVT) mRNA under various conditions, including varying types and degrees of stress conditions, and methods for preventing the degradation of mRNA under various conditions, including heat exposure.

### BACKGROUND OF THE INVENTIONS

Messenger RNAs (mRNAs) are large complex polyanionic polymers that provide the template for production of proteins within all living cells and represent a new class of therapeutic molecules for treating various diseases. RNA therapeutics have provided new options for the prevention of diseases caused by emerging pathogens, cancers, and other genetic disorders. While the concept of nucleic acid-based medicines has been considered for several decades, various challenges prevented the practical application of RNA therapeutics, such as the instability of the RNA molecule, the immunostimulatory effects of RNA against innate immune pathways, the difficulty of and delivery of molecules into cells. Recent innovations have improved the stability profiles and delivery of different RNA formats which, in turn, have enabled the development of various new types of nucleic acid therapeutics. In addition to the approval of these new drugs, the 2020 Emergency Use Authorization by the Food and Drug Administration (FDA) for Comirnaty (Pfizer-BioNTech) and Spikevax (Moderna) mRNA vaccines for the prevention of coronavirus disease advanced mRNA technologies into the spotlight (FDA Takes Additional Action in Fight Against COVID-19 by Issuing Emergency Use Authorization for Second COVID-19 Vaccine, FDA.Gov. (2020)). The rapid growth of mRNA therapeutics at the research and development stages and then later into the clinic presents several new challenges and opportunities with respect to manufacturing, characterization, and evaluation of critical quality attributes (CQAs) (Patel, et al., Characterization of BNT162b2 mRNA to Evaluate Risk of Off-Target Antigen Translation, J. Pharm. Sci. 112 (2023) 1364-1371; Poveda, et al., Establishing Preferred Product Characterization for the Evaluation of RNA Vaccine Antigens, Vaccines. 7 (2019) 131).

The inherent instability of RNA is rooted in its chemical structure, and the presence of a reactive hydroxyl group at the 2′ position of the ribose ring. This reactive moiety participates in acid-, base-, and metal-catalyzed hydrolysis of the sugar-phosphate backbone, causing fragmentation of the molecule (AbouHaidar and Ivanov, Non-Enzymatic RNA Hydrolysis Promoted by the Combined Catalytic Activity of Buffers and Magnesium Ions, Z. Für Naturforsch. C. 54 (1999) 542-548). The single-stranded nature of RNA further increases its chemical susceptibility to stresses, including UV-induced and oxidation-mediated strand-scission. While local double-stranded and structured regions do exist in mature RNA transcripts, the many loops, bulges, and single-stranded stretches render the molecule vulnerable to chemical assaults and other environmental stressors. Furthermore, unlike DNA, whose structure is reinforced by a stable double-helix, RNA lacks uniform structure and instead participates in a variety of non-canonical interactions enabling it to be widely dynamic in response to temperature fluctuations and other environmental conditions (Ganser, et al., The roles of structural dynamics in the cellular functions of RNAs, Nat. Rev. Mol. Cell Biol. 20 (2019) 474-489; Vicens and Kieft, Thoughts on how to think (and talk) about RNA structure, Proc. Natl. Acad. Sci. 119 (2022)). Forced degradation studies challenge the stability of new molecules, under conditions encountered during typical development-related unit operations and are important in defining candidate robustness and likely degradation pathways. The knowledge gained from stress studies can aid in the optimization and selection of formulation conditions and inform on storage conditions and shelf life with the goal of maximizing stability. Furthermore, forced degradation, as disclosed herein, help to confirm that quantitative analytical methods are stability-indicating and exhibit the sensitivity to detect aggregation and breakdown products. Unlike protein biologics, which are produced in cells, and other oligonucleotide therapeutics, such as small interfering RNAs and antisense oligonucleotides, which are synthesized using solid-phase chemistry, mRNA vaccines can be produced using a cell-free approach from a DNA template and T7 RNA polymerase-driven in vitro transcription (IVT) methods (Beckert and Masquida, Synthesis of RNA by in vitro transcription., Methods Mol. Biol. (Clifton, NJ) 703 (2010) 29-41). In addition to their unique production schemes, mRNAs are comprised of structural features and chemical components that are not present in other oligonucleotide therapeutics. These elements chemically and structurally differentiate mRNA products from other existing modalities and necessitate a concise panel of assays that are suitable for investigating their integrity, aggregation, and degradation products. Although agencies have published guidance documents and proposed regulatory requirements for mRNA vaccine quality, analytical procedures, and proposed studies, an industry standard workflow has not yet been adopted (Cheng, et al., Research Advances on the Stability of mRNA Vaccines, Viruses. 15 (2023) 668).

Divalent metals participate in a wide array of cellular functions, and can affect RNA. Previous studies measured the concentration of magnesium in Escherichia coli to be about 54 mM; however, studies have demonstrated that the concentration of free magnesium was as low as 1.5-3.0 mM in bacteria and 0.5-1.0 mM in eukaryotic cells. Research as early as the 1960s investigated the binding constants of citrate complexes with cellular metals, the significance of which was not known at the time, and presently helps to rationalize the discrepancy between free and associated magnesium in cells. Later, the Szostak lab demonstrated that citrate could chelate Mg²⁺ and minimize degradation of single-stranded RNA, and a more recent study demonstrated that amino-acid chelation of magnesium cations also stabilizes RNA against magnesium-dependent hydrolysis (Adamala and Szostak, Nonenzymatic Template-Directed RNA Synthesis Inside Model Protocells, Science. 342 (2013) 1098-1100). Today, chelating agents are used as additives in preparative buffers during routine laboratory activities, and several protocols employ EDTA as a stabilizer in the preparation of DNA samples for downstream analysis (Nkuna, et al., Applying EDTA in Chelating Excess Metal Ions to Improve Downstream DNA Recovery from Mine Tailings for Long-Read Amplicon Sequencing of Acidophilic Fungi Communities, J. Fungi. 8 (2022) 419).

In view of the needs for characterizing mRNA, the present inventions provide efficient modalities for evaluating mRNA and assessing critical quality attributes (CQAs).

### SUMMARY OF THE INVENTIONS

The inventions provide methods of evaluating critical quality attributes (CQAs) of messenger ribonucleic acid (mRNA) molecules in a sample, wherein the method comprises the steps of: (a) determining the purity of the mRNA contained in the sample using ion-paired reverse-phase (IPRP) liquid chromatography; (b) determining the size distribution of the mRNA contained in the sample using size exclusion chromatography (SEC) for separation of different size molecules, and multi-angle light scattering (MALS) detector to determine the molar mass of the SEC eluted sample, thereby identifying species size distribution in the sample; and (c) comparing the purity of the mRNA from step (a) and the SEC-MALS species size distribution from step (b) to intact mRNA, thereby evaluating the CQAs of the mRNA molecules present in the sample. The intact mRNA data are from mRNA at Day 0 (D0) for experimental mRNA (for example, see FIGS. 11A-11B and 22A-22H) or fresh sample of mRNA that was not under any stress condition. According to the inventions an ultraviolet light (UV) detector or a refractive index (RI) detector can be used at step (b). The mRNA can be further evaluated for 5′ cap integrity by enzymatic digestion of the sample in combination with the SEC. The mRNA can be further evaluated for 3′ poly(A) integrity by oligo(dT) affinity capture. The mRNA can be analysed for size distribution, wherein the size distribution is determined by SEC-MALS (SEC-MALLS). The CQAs can be selected from the group consisting of mRNA purity; mRNA integrity including 5′ cap integrity and 3′ poly(A) integrity; and impurities including mRNA aggregates, mRNA fragments, DNA, peptide or polypeptide, nucleoside triphosphates (NTPs), solvents, and dsRNA. According to the inventions the mRNA samples are preserved under various stress and storage conditions, such as heat, humidity, pressure, light, and the like.

The inventions also provide methods for analyzing the condition of mRNA molecules in a sample, wherein the method comprises the steps of: (a) determining the purity of the mRNA contained in the sample using ion-paired reverse-phase (IPRP) liquid chromatography; (b) determining the size distribution of the mRNA contained in the sample using size exclusion chromatography (SEC) for separation of different size molecules, and multi-angle light scattering (MALS) detector to determine the molar mass of the SEC eluted sample, thereby identifying species size distribution in the sample; and (c) comparing the purity of the mRNA from step (a) and the SEC-MALS species size distribution from step (b) to intact mRNA, thereby analyzing the condition of the mRNA molecules present in the sample. The intact mRNA data are from mRNA at D0 for experimental mRNA (for example, see FIGS. 11A-11B and 22A-22H) or fresh sample of mRNA that was not under any stress condition. According to the inventions an ultraviolet light (UV) detector or a refractive index (RI) detector can be used at step (b). The mRNA can be further evaluated for 5′ cap integrity by enzymatic digestion of the sample in combination with the SEC. The mRNA can be further evaluated for 3′ poly(A) integrity by oligo(dT) affinity capture. The mRNA can be analysed for size distribution, wherein the size distribution is determined by SEC-MALS or SEC-MALLS. The mRNA can be preserved under various stress conditions. The migration time and size of peaks are compared, wherein the migration time can be a relative migration time. The mRNA sample of step (a) can shows a reduction in polyadenylated (poly(A)) mRNA species.

The inventions also provide characterized mRNA samples obtained according to any of the methods disclosed herein.

The inventions further provide systems of evaluating critical quality attributes (CQAs) of messenger ribonucleic acid (mRNA) molecules in a sample, wherein the system comprises: (a) ion-paired reverse-phase (IPRP) liquid chromatography for determining the purity of the mRNA contained in the sample; (b) size exclusion chromatography (SEC) for determining the size distribution of the mRNA contained in the sample for separation of different size molecules, and multi-angle light scattering (MALS) detector to determine the molar mass of the SEC eluted sample, thereby identifying species size distribution in the sample; and (c) comparing the purity of the mRNA from step (a) and the SEC-MALS species size distribution from step (b) to intact mRNA, thereby evaluating the CQAs of the mRNA molecules present in the sample. The intact mRNA data are from mRNA at D0 for experimental mRNA (for example, see FIGS. 11A-11B and 22A-22H) or fresh sample of mRNA that was not under any stress condition. The intact mRNA data are from mRNA at D0 for experimental mRNA (for example, see FIGS. 11A-11B and 22A-22H) or fresh sample of mRNA that was not under any stress condition. According to the inventions an ultraviolet light (UV) detector or a refractive index (RI) detector can be used at step (b). The mRNA can be further evaluated for 5′ cap integrity by enzymatic digestion of the sample in combination with the SEC. The mRNA can be further evaluated for 3′ poly(A) integrity by oligo(dT) affinity capture. The mRNA can be analysed for size distribution, wherein the size distribution is determined by SEC-MALS or SEC-MALLS. The CQAs can be selected from the group consisting of mRNA purity; mRNA integrity including 5′ cap integrity and 3′ poly(A) integrity; and impurities including mRNA aggregates, mRNA fragments, DNA, peptide or polypeptide, NTPs, solvents, and dsRNA. The mRNA can be preserved under various stress and storage conditions, such as heat, humidity, pressure, light, and the like.

The inventions further provide methods of minimizing the degradation of mRNA during heat exposure comprising adding EDTA to a mRNA preparation prior to heat exposure, wherein the degradation of mRNA is determined by analyzing the mRNA molecules in a sample, wherein the method comprises the steps of: (a) determining the purity of the mRNA contained in the sample using ion-paired reverse-phase (IPRP) liquid chromatography; (b) determining the size distribution of the mRNA contained in the sample using size exclusion chromatography (SEC) for separation of different size molecules, and multi-angle light scattering (MALS) detector to determine the molar mass of the SEC eluted sample, thereby identifying species size distribution in the sample; and (c) comparing the purity of the mRNA from step (a) and the SEC-MALS species size distribution from step (b) to intact mRNA, thereby analyzing the condition of the mRNA molecules present in the sample. The intact mRNA data are from mRNA at D0 for experimental mRNA (for example, see FIGS. 11A-11B and 22A-22H) or fresh sample of mRNA that was not under any stress condition. According to the inventions an ultraviolet light (UV) detector or a refractive index (RI) detector can be used at step (b). The mRNA can be further evaluated for 5′ cap integrity by enzymatic digestion of the sample in combination with the SEC. The mRNA can be further evaluated for 3′ poly(A) integrity by oligo(dT) affinity capture. The mRNA can be analysed for size distribution, wherein the size distribution is determined by SEC-MALS or SEC-MALLS. The mRNA can be preserved under various stress conditions. The migration time and size of peaks are compared, wherein the migration time can be a relative migration time. The mRNA sample of step (a) can shows a reduction in polyadenylated (poly(A)) mRNA species. The mRNA can be exposed to a temperature of about 35° C. to about 65° C. for up to 10 days. The mRNA can be exposed to a temperature of about 40° C. for up to 6 days. The mRNA can be exposed to a temperature of about 40° C. for up to 6 days. The concentration of mRNA can be about 0.01 mg/mL to 100 mg/mL. The mRNA can be treated with about 0.01 mM to about 100 mM RNase-free EDTA.

### BRIEF DESCRIPTION OF THE FIGURES

FIGS. 1A and 1B schematically depict the anatomy of a messenger RNA (mRNA) transcript. FIG. 1A schematically displays key structural features of an mRNA. 5′ methyl-7-guanine cap (5′ Cap) is shown as a pentagon. FIG. 1B schematically displays a “closed-loop model” of translation initiation. Cap structure is recognized by eukaryotic translation initiation factor 4E (eIF4E) while poly(A)-binding protein (PABP) binds to the polyadenine (poly(A)) tail. eIF4E and PABP assemble onto eukaryotic translation initiation factor 4G (eIF4G) scaffold, leading to eukaryotic translation initiation factor 4A (eIF4A)-assembly and recruitment of ribosomal machinery.

FIG. 2 schematically depicts experimental design for 6-day stress study and characterization of mRNAs. All four transcripts were incubated at 40° C. for 6 days. Aliquots were removed at the starting time (D0) and on each day (D1, D2, D3, D5, and D6). Following the heat incubation, all samples were collected and analyzed for their quality attributes by using the methods shown.

FIGS. 3A to 3G schematically depict the results of evaluating of mRNA purity by denaturing ion-paired reverse-phase (IPRP) liquid chromatography. FIGS. 3A to 3D are graphs showing individual chromatograms for each transcript (EGFP, OVA, FLuc and Cas9). FIGS. 3E and 3F are graphs showing D0 to D6 chromatograms for EGFP and Cas9 transcripts, respectively. Data were fitted to a single-phase decay model for calculation of half-life. FIG. 3G depicts quantification of 6-day stability profiles for all four transcripts (EGFP, OVA, FLuc and Cas9).

FIGS. 4A to 4D schematically depict size exclusion chromatography-multi-angle light scattering (SEC-MALS) analysis of transcripts and calculation of molecular weight. D0 and D6 SEC-MALS chromatograms are shown for each transcript (EGFP (FIG. 4A), OVA (FIG. 4B), FLuc (FIG. 4C) and Cas9 (FIG. 4D)). Molar masses were obtained by measuring refractive index and calculating mass using a dn/dc_{mRNA}=0.172 mL/g. Theoretical mass for each mRNA was calculated from the transcript length provided by the manufacturer assuming about 120 nt poly(A) signal and an average of 330 Da/nucleotide. 1,145 Da for CleanCap 5′ cap structure was added to each theoretical mass. EGFP_{theoretical mass≈}330 kDa (+/−16.5 kDa), OVA_{theoretical mass≈}476 kDa (+/−24 kDa), FLuc_{theoreticalmass=≈}638 kDa (+/−32 kDa), Cas⁹_{theoreticalmass≈}1493 kDa (+/−75 kDa). A 5% variation in theoretical mass is shown to account for instrument variability disclosed by vendor.

FIGS. 5A and 5B schematically depict 5′ capping analysis of transcripts using sequential enzymatic digestion followed by SEC analysis. FIG. 5A schematically outlines the procedure for evaluating the presence of 5′ cap. FIG. 5B depicts quantification of the capping percentages for each transcript at D0 and D6.

FIGS. 6A and 6B schematically outline the procedure for determining poly(A) presence using an oligo(dT) affinity capture. FIG. 6A is a schematic of 3′ poly(A) capture assay. Transcripts lacking a poly(A) tail are unable to bind to the column and are collected in the flow through fraction. Transcripts with partially intact poly(A) tails are weakly bound to the column and are removed during the wash step. Intact poly(A) tails allow the transcript to tightly bind the oligo(dT) column and come off only when elution buffer is added. FIG. 6B are graphs depicting UPLC profiles of Cas9 mRNA on the oligo(dT) column at D0 and D6.

FIGS. 7A to 7D are IPRP chromatograms demonstrating the protective impact of EDTA during post-transcriptional processes. FIGS. 7A and 7B depicting IPRP chromatogram of Cas9 mRNA populations resolved by denaturing IPRP at D0 and D6, respective, without the addition of EDTA. FIGS. 7C and 7D depicting IPRP chromatogram of Cas9 mRNA at D0 and D6, respectively, when EDTA is added to the incubation buffer.

FIGS. 8A and 8B are graphs depicting the mRNA purity profiles of the transcripts as determined by denaturing IPRP liquid chromatography. FIG. 8A is magnified in FIG. 8B. Superposition of the four profiles shows relative retention to stationary phase.

FIGS. 9A to 9D are graphs depicting individual 6-day IPRP chromatograms for each transcript. D0 to D6 IPRP profiles for each transcript show the main peak diminishing over time.

FIG. 10 is a bar graph depicting quantification of low molecular weight (LMW) populations as detected in SEC-MALS analysis. The peak area of the LMW population was quantified at D0 and D6. Increase in transcript length is mirrored by an increase in LMW population as open reading frame (ORF) length increases.

FIGS. 11A and 11B are graphs showing representative 5′ Capping analysis SEC chromatograms. FIG. 11A shows EGFP D0 sample 5′ capping efficiency analysis and FIG. 11B shows Cas9 D0 sample 5′ capping efficiency analysis. Samples were either treated with enzymes for capping analysis or untreated. Samples are resolved by SEC, and percent (%) of capped mRNA is back calculated from the difference in peak area between the treated and untreated samples.

FIGS. 12A and 12B are graphs showing representative chromatograms for oligo-(dT) affinity capture for EGFP and FLuc mRNA, respectively.

FIGS. 13A and 13B are graphs showing SEC-MALS of Cas9 mRNA or Cas9 with EDTA, respectively. D0 and D6 SEC-MALS chromatograms are shown for each Cas9 mRNA either in the absence (FIG. 13A) or presence (FIG. 13B) of EDTA.

FIG. 14 is schematically depicting a summary of analytical methods for mRNA quality attributes concerning mRNA identity, purity and integrity.

FIG. 15 provides a summary of analytical methods for mRNA quality attributes concerning mRNA characterization and impurities.

FIGS. 16A to 16D illustrate an overview of respective EGFP, OVA, FLuc and Cas9 mRNA constructs used for evaluations.

FIG. 17 schematically depicts simplified thermal stress experiments for mRNA degradation study using EGFP, OVA, FLuc and Cas9 RNA.

FIGS. 18A to 18F are graphs showing mRNA Integrity as analyzed by ion-paired reverse-phase—liquid chromatography (IPRP-LC). FIG. 18A is magnified in FIG. 18D. FIGS. 18B, 18C, 18E and 18F showing individual chromatograms for each transcript EGFP, FLuc, OVA, and Cas9, respectively.

FIGS. 19A to 19F are graphs showing different integrity profiles of EGFP (FIGS. 19A-19C) and Cas9 (FIGS. 19D-19F) under thermal stress.

FIGS. 20A to 20D are graphs showing that LMW species respectively of EGFP, OVA, FLuc, and Cas9 increase with thermal stress for mRNA as detected in SEC-MALS analysis.

FIGS. 21A and 21B schematically depict capping analysis of transcripts using sequential enzymatic digestion followed by SEC analysis. FIG. 21A schematically outlines the procedure for evaluating the presence of 5′ cap under thermal stress (see also FIG. 5A). FIG. 21B is a depicts quantification of the normalized 5′ capping percentages for each transcript at D0 and D6 and showing that 5′ Cap stayed intact during the thermal stress.

FIGS. 22A to 22H are graphs showing 5′ capping analysis SEC chromatograms for all four mRNA samples in the following order: EGFP, OVA, FLuc and Cas9 D0 or D6 samples. Samples were either treated with enzymes for capping analysis or untreated.

FIGS. 23A to 23C: Uridine bases of in vitro transcribed mRNA constructs, as disclosed herein, can be modified as UTR (FIG. 23A), 5moU (FIG. 23B) and N1U (FIG. 23C) and for analysis by SEC-MALS.

FIGS. 24A to 24F: FIGS. 24A, 24B and 24C depicting initial SEC-MALS data collection for EGFP constructs with uridine modified as UTR, 5moU and N1U, respectively. Data showing varying levels of high molecular weight/aggregate species. FIGS. 24D, 24E and 24F depicting SEC-MALS data for EGFP constructs with uridine modified as UTR, 5moU and N1U, respectively, that have been treated with a temperature mediated unfolding/refolding step. Data showing resulted disruption of the larger aggregates.

FIGS. 25A to 25F: FIGS. 25A, 25B and 25C depicting initial SEC-MALS data collection for OVA constructs with uridine modified as UTR, 5moU and N1U, respectively. Data showing varying levels of high molecular weight/aggregate species. FIGS. 25D, 25E and 25F depicting SEC-MALS data for OVA constructs with uridine modified as UTR, 5moU and N1U, respectively, that have been treated with a temperature mediated unfolding/refolding step. Data showing resulted disruption of the larger aggregates.

FIGS. 26A to 26F: FIGS. 26A, 26B and 26C depicting initial SEC-MALS data collection for FLuc constructs with uridine modified as UTR, 5moU and N1U, respectively. Data showing varying levels of high molecular weight/aggregate species. FIGS. 26D, 26E and 26F depicting SEC-MALS data for FLuc constructs with uridine modified as UTR, 5moU and N1U, respectively, that have been treated with a temperature mediated unfolding/refolding step. Data showing resulted disruption of the larger aggregates.

FIGS. 27A to 27F: FIGS. 27A, 27B and 27C depicting initial SEC-MALS data collection for Cas9 constructs with uridine modified as UTR, 5moU and N1U, respectively. Data showing varying levels of high molecular weight/aggregate species. FIGS. 27D, 27E and 27F depicting SEC-MALS data for Cas9 constructs with uridine modified as UTR, 5moU and N1U, respectively, that have been treated with a temperature mediated unfolding/refolding step. Data showing resulted disruption of the larger aggregates.

### DETAILED DESCRIPTION OF THE INVENTIONS

### Definitions

Unless defined otherwise, all technical and scientific terms used herein have the same meaning as commonly understood by one of ordinary skill in the art to which these inventions belong.

The term “about” in the context of numerical values and ranges refers to values or ranges that approximate or are close to the recited values or ranges such that the inventions can perform as intended, such as having a desired rate, amount, density, degree, increase, decrease, percentage, value, purity, pH, concentration, presence of a form or variant, temperature or amount of time, as is apparent from the teachings contained herein. For example, “about” can signify values either above or below the stated value in a range of approx. +/−10% or more or less depending on the ability to perform. Thus, this term encompasses values beyond those simply resulting from systematic error.

“Purification” in its various grammatical forms includes, but is not limited to, the use of one or more procedures, such as depth filtration, tangential flow filtration, affinity capture, ionic exchange (such as anionic exchange and cationic exchange) and the like.

The term “messenger RNA (mRNA)”, as used herein, refers to a polyribonucleotide that encodes at least one polypeptide. Messenger RNA, as used herein, can encompass both modified and unmodified mRNA and can contain one or more coding and non-coding regions. Messenger RNA can be obtained, purified, or isolated from naturally occurring sources, including any biological sources, can be produced using recombinant expression systems, in vitro transcribed, or chemically synthesized. Messenger RNA can include a nucleotide sequence having a coding region that codes for a polypeptide, a 5′ untranslated region (5′ UTR) upstream of the coding region, a 3′untranslated region (3′ UTR) downstream of the coding region, a cap at the 5′ terminus and a polyA or polyadenylation region downstream of the 3′UTR. Generally, in eukaryotic organisms, mRNA processing comprises transcription of the mRNA from DNA and the addition of a “cap” on the N-terminal (5′) end, and a “tail” on the C-terminal (3′) end. Generally, a cap is a 7-methylguanosine cap, which is a guanosine that is linked through a 5′-5′-triphosphate bond to the first transcribed nucleotide. The tail is typically a polyadenylation event whereby a polyadenylyl moiety is added to the 3′ end of the mRNA molecule. The “tail” serves to protect the mRNA from exonuclease degradation.

As used herein, the term “naturally occurring” refers to materials which are found in nature or a form of the material that is found in nature.

As used herein, the term “polynucleotide” refers to a polymer of nucleosides. Typically, a polynucleotide is composed of nucleosides that are naturally found in DNA or RNA (for example, adenosine, thymidine, guanosine, cytidine, uridine, deoxyadenosine, deoxythymidine, deoxyguanosine, and deoxycytidine) joined by phosphodiester bonds. The term encompasses molecules comprising nucleosides or nucleoside analogs containing chemically or biologically modified bases, modified backbones, etc., whether or not found in naturally occurring nucleic acids, and such molecules may be preferred for certain applications. Where this application refers to a polynucleotide it is understood that both DNA, RNA, and in each case both single- and double-stranded forms (and complements of each single-stranded molecule) are provided. “Polynucleotide sequence” as used herein can refer to the polynucleotide material itself and/or to the sequence information (that is, the succession of letters used as abbreviations for bases) that biochemically characterizes a specific nucleic acid. A polynucleotide sequence presented herein is presented in a 5′ to 3′ direction unless otherwise indicated.

The term “MALS”, as used herein, is an abbreviation for “multi-angle light scattering”, refers to a technique using instrumentation that allows characterization and determination of molecular mass of a sample. Likewise, the term “MALLS”, as used herein, is an abbreviation for “multi-angle laser light scattering”. These terms often are used interchangeably in the field. Characterization of small particles for properties such as size, mass, shape, as well as the associated distributions of these quantities within a sample solution, has been the objective of a broad range of analytical instruments. Light scattering instrumentation plays a major role among them as the technique does not require calibration standards, which is extremely useful in characterizing small particles such as molecules, DNA, RNA, viruses, and other classes of nano-particles. The ability to measure the size distributions of the mass present in a scattering sample has been of particular importance. To assess these distributions, it is necessary to separate the particles present in the sample so that the scattering properties and the concentration of each separated species present can be measured separately. The separation has been achieved traditionally by processes referred to as chromatographic separation. A combination of MALS (MALLS) with size-exclusion chromatographic (SEC) separation and molar mass/concentration measurement can allow the determination of these size distributions. Further, interfacing SEC separation with MALS or MALLS can allow determination of the molar mass of an eluting sample, provide a deeper characterization of the different populations in the sample.

All numerical limits and ranges set forth herein include all numbers or values thereabout or there between of the numbers of the range or limit. The ranges and limits described herein expressly denominate and set forth all integers, decimals and fractional values defined and encompassed by the range or limit. Thus, a recitation of ranges of values herein are merely intended to serve as a way of referring individually to each separate value falling within the range, unless otherwise indicated herein, and each separate value is incorporated into the specification as if it were individually recited herein.

### Description

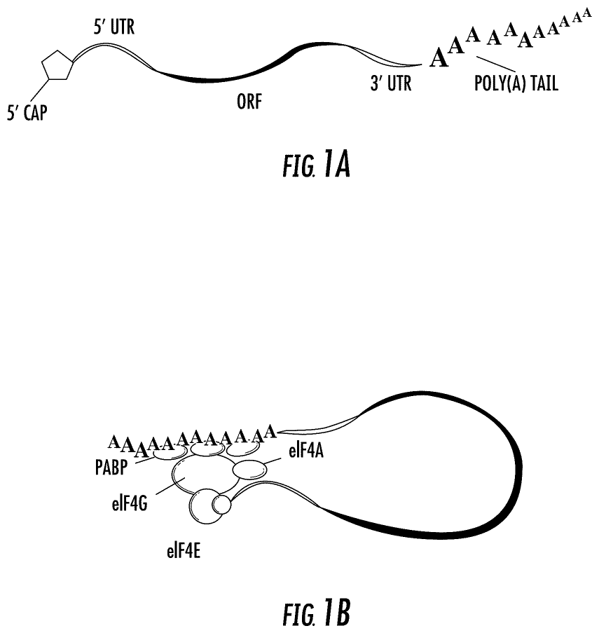

Messenger RNA (mRNA) is comprised of a 5′ cap structure, an open reading frame (ORF) corresponding to the gene of interest flanked by 5′ and 3′ untranslated regions (UTRs), and a terminal 3′ polyadenine (poly(A)) tail (FIG. 1A). The ‘closed-loop’ model of translation illustrates how protein production begins when translation components jointly assemble on the 5′ cap and 3′ poly(A) tail. Eukaryotic translation initiation factor 4E (eIF4E) recognizes and binds to the 5′ cap structure while poly(A)-binding protein (PABP) decorates the poly(A) tail. eIF4G bridges PABP on the poly(A) tail to the 5′ cap and eIF4E, ultimately leading to ribosome assembly and subsequent protein synthesis (FIG. 1B). Chemical alteration or degradation of these structural components can negatively impact product safety and efficacy. The ability to enhance mRNA half-life by amending structural features at the template level or by incorporating nucleotide analogs has improved the stability and translation efficiency of mRNA vaccines. For example, improvements to the design of the coding region by optimizing codon usage can enhance protein production with the selection of codons that diversify and more economically use the pool of available transfer RNAs or that increase GC-content within the ORF to bolster structural stability. With respect to innate immune activation, the finding that certain modifications to uridine prevent activation of a Toll-like receptor (TLR)-response further widened the opportunities for the incorporation of various synthetic nucleotides, including pseudouridine and 5′methoxyuridine which were shown to evade Protein Kinase R (PKR) activation and interferon beta (IFN-β) secretion, respectively. The use of synthetic cap analogs during transcription ensures the incorporation of a mature cap structure mimicking the output of cellular capping mechanisms, an improvement over traditional post-transcriptional multi-enzyme processes commonly used to cap IVT mRNA. This development not only reduces process steps but also eliminates the risk of immature, unmethylated cap structures being detected by innate immune sensors. Improvements to these elements continue to expand and facilitate advances in the field of mRNA vaccines and therapeutics.

The present inventions provide a concise panel of stability-indicating methods that can be used to rapidly monitor the critical quality attributes (CQAs) of in vitro transcription (IVT) mRNAs. These methods are intended to be basic yet sensitive enough to detect a wide range of impurities accumulated following exposure to environmental stress. To demonstrate the utility of the chromatography-based strategy of the current invention, heat was employed as a stressor and evaluated the stabilities of four commercially available mRNA constructs that are frequently used in both academic and industrial settings. The four transcripts, EGFP-, OVA-, FLuc-, and Cas9-encoding mRNAs, all similarly share a 5′Cap1-like analogue, a 120 nt poly(A) tail, and were transcribed using 5-methoxyuridine (5moU) in place of uridine. However, all vary in the length of their ORF, yielding final constructs ranging from about 1,000-4,000 nts (Henderson, et al., Cap 1 Messenger RNA Synthesis with Co-transcriptional CleanCap® Analog by In Vitro Transcription, Curr. Protoc. 1 (2021) e39) (Table 1). All transcripts harbor a Cap1-type CleanCap capping analog and were synthesized with 5moU modified nucleotides.

TABLE 1
Description of mRNA transcripts used in this study.

| | | Poly(A) tail | Expected molar |
|---|---|---|---|
| mRNA | Length (nt) | length (nt)* | mass (kDa)** |
| EGFP | 996 | 120 | 330 |
| OVA | 1,437 | 120 | 476 |
| FLuc | 1,929 | 120 | 638 |
| Cas9 | 4,521 | 120 | 1,493 |

*Poly(A) tail length is reported as an average length provided by the supplier.
**Estimated molar mass is calculated by: (# nucleotides × 330 Da) + 1145 kDa for 5′ cap.

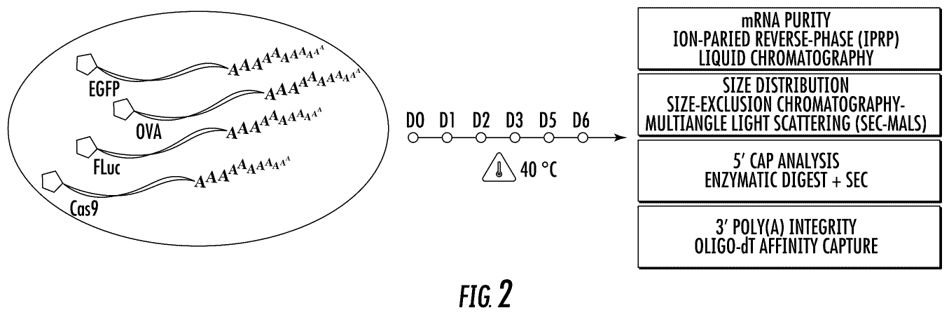

The characterization assessment following thermal stress at 40° C. for 6 days focuses on the most relevant factors of the mRNA that are critical for translation of the target protein. These factors include 1) total mRNA purity, 2) size distribution, 3) presence of the 5′ cap structure, and 4) integrity of 3′ poly(A) tail (FIG. 2).

Heightened interest in messenger RNA (mRNA) therapeutics has accelerated the need for analytical methodologies that facilitate the production of supplies for clinical trials. Forced degradation studies are routinely conducted to provide an understanding of potential weak spots in the molecule that are exploited by stresses encountered during bulk purification, production, shipment, and storage. Consequently, temperature fluctuations and excursions are often experienced during these unit operations and may accelerate mRNA degradation.

Stress studies are critical to the drug development process and help ensure that analytical assays are stability-indicating. The present inventions characterize the stability of four commercially available transcripts during thermal stress by implementing routine analytical chromatography procedures. Purity of the four transcripts was first evaluated using IPRP liquid chromatography under denaturing conditions, a well-established method for the analysis of RNA impurities (Azarani and Hecker, RNA analysis by ion-pair reversed-phase high performance liquid chromatography, Nucleic Acids Res. 29 (2001) e7-e7). Examination of the total mRNA purity provided a comparison of the main peak purity across samples as well as the relative amounts of pre-main and post-main populations made up of degradation products and long-mers (a post-main peak where longer impurities, often termed “long-mers” or high molecular weight (HMW) impurities), respectively. The present inventions revealed that the longer transcripts were more susceptible to degradation as demonstrated by the elevated degradation of Cas9 compared to EGFP mRNA. This can be attributed to the higher number of sugar-phosphate linkages in the longer transcripts, as each additional linkage serves as a substrate for contaminating nuclease or site for hydrolysis. This observation is consistent with cellular data showing that mRNA half-life was negatively correlated with transcript length, so it is therefore expected that Cas9 mRNA was more readily degraded than its shorter counterparts. This trend was maintained across all four transcripts tested, where the calculated half-lives inversely trended with the number of bases (Table 2).

TABLE 2
Calculated half-life of each transcript*.

| mRNA | mRNA length (nt) | Half-life (days) |
|---|---|---|
| EGFP | 996 | 11 |
| OVA | 1,437 | 5.1 |
| FLuc | 1,929 | 3.3 |
| CAS9 | 4,521 | 1.9 |

*Half-lives were calculated from IPRP data by plotting Main peak area over time and fitting the data to a single-phase decay model.

The longer transcripts also can be difficult to produce based on the initial purity of the starting material. While the EGFP starting material was about 95% pure, the corresponding analysis of Cas9 revealed it to be only about 50% pure.

Together, the size exclusion chromatography (SEC) and ion-paired reverse-phase (IPRP) experiments provide considerable information regarding the populations within a sample and their relative size and integrity. In line with United States Pharmacopoeia (USP) draft guidelines for mRNA vaccine quality analytical procedures, analytical IPRP and SEC analyses were employed for quantifying the percentage of fragmented mRNA and aggregation, respectively. However, without proper size markers it is difficult to conclude with certainty that even the main peak population under native conditions corresponds to the appropriate mass and the expected full-length product. Appropriate markers for folded RNAs are currently unavailable since they fold in unpredictable and complicated ways in solution. Moreover, given the dynamic nature of RNA folding, RNA molecules that differ in length may share an apparent size and hydrodynamic radius, and thus may not adequately serve as size standards. Consequently, SEC coupled to a multi-angle light scattering (MALS) detector was employed to assess the molar mass of each population as it eluted from the column. The calculated molar mass of the main peak for each mRNA transcript confirmed the predominant species in solution was consistent with an intact mRNA transcript. Furthermore, when utilizing UV absorbance or refractive index (RI) as a concentration source, MALS can be coupled to various liquid chromatography-based separation techniques to determine the molar mass and size distribution within the sample regardless of elution behavior of the molecule.

Although the purity evaluation by IPRP and SEC-MALS demonstrated that the transcripts degraded over time at elevated temperatures, these methods did not indicate whether the 5′ cap or the 3′ poly(A) tail were more prone to instability. The 5′ cap and 3′ poly(A) analyses allow for quick and reliable evaluation of the integrity of critical structural components using straightforward protocols while consuming little sample. When performed in parallel, these methods provide information about which end of the transcript, if either, enabled the degradation pathway. This information can be especially useful when comparing the incorporation efficiency of different capping analogs or evaluating the impact of alterations to the UTRs and poly(A) designs. Assays similar to the USP draft guidelines were used with some modifications to improve robustness. For quantifying the percentage of capped mRNA, the USP guidance recommends using an RNase H-based strategy to cleave the transcript and chromatographically resolve 5′ capped and uncapped fragments. This RNase H method relies on generating sequence-specific probes antisense to the mRNA sequence for each construct, limiting the application of this method as platform. Further, the RNase H method requires that probes can access and anneal to the mRNA as a prerequisite to RNase H cleavage, adding an additional step whose efficiency may vary across constructs and sequences. An alternative to the RNase H strategy, the procedures applied herein use 5′-specific enzymes that sequentially breakdown uncapped RNAs by recognizing 5′ terminal groups. The use of these enzymes makes the assay more specialized for 5′ cap evaluation and can be universally applied to all transcripts regardless of 5′UTR or ORF sequence. This alternative to RNase H also reduces time and costs lost to probe design and synthesis. Under the thermal stress conditions examined, the 5′ cap integrity remained largely unchanged over the incubation period, highlighting the stability of the cap analog used in the preparation of these transcripts. More aggressive stress conditions can be done to destabilize the 5′ terminus or to further fragment the entirety of the transcript to observe more impurities near the 5′ end.

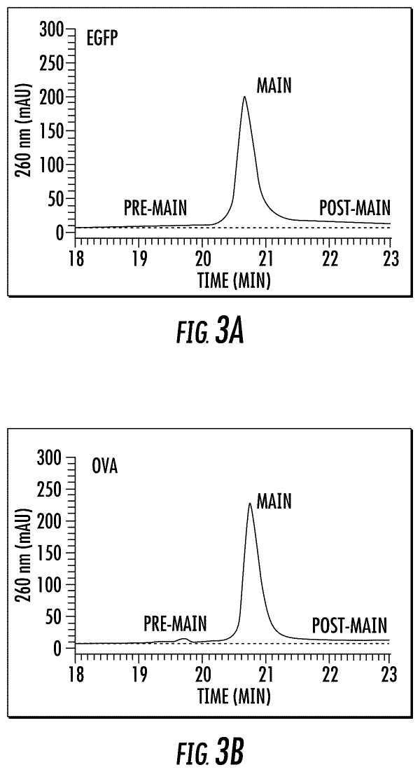

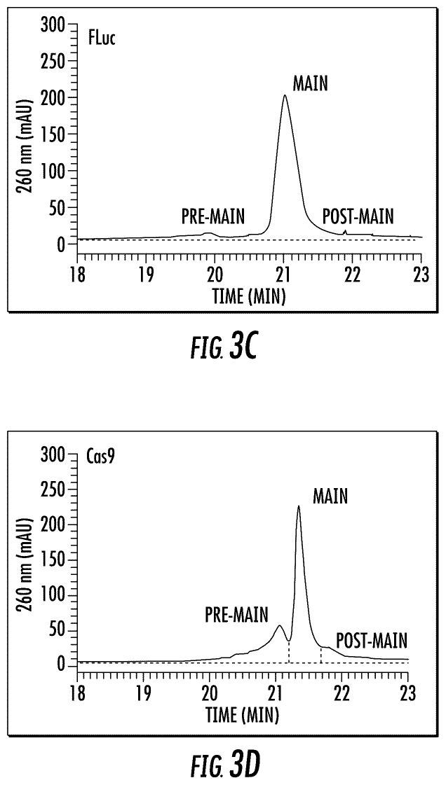

Oligo-(dT) affinity capture was used to estimate the percentage of transcripts retaining 3′ poly(A) tails. Similar to the capping analysis, USP draft guidance recommends the use of antisense probes that anneal to the 3′ end of the ORF and free the poly(A) tail after RNase H cleavage. This approach is subject to the same aforementioned limitations of RNase H methods. The advantage of the oligo-(dT) affinity capture column used here allows a universal strategy for all transcripts and is specific to the analysis of poly(A) tails, as it is dependent on the strength of the poly(A):poly(dT) interaction. This method revealed that EGFP, OVA, and FLuc displayed relatively similar levels of intact poly(A) tail preservation, degrading by about 7-10% each at day 6, whereas the 3′ end of the considerably longer Cas9 transcript degraded by about 30%. Importantly, as orthogonally demonstrated by the total mRNA purity analysis (FIGS. 3A-3G), the Cas9 (see FIGS. 3D, 3F and 3G) mRNA starting material was initially far less pure compared with the three smaller transcripts. This emphasizes not only the importance of the poly(A) tail from shielding the ORF from exonucleases, but also the general challenges associated with transcription of longer ORFs and their impact on processes and production.

Thermal stress data of four mRNA transcripts presented herein that EDTA reduced Cas9 mRNA breakdown that was likely due to the presence of trace divalent metal ions in the mRNA samples. Trace divalent metal contamination that can accelerate RNA instability is likely carried over from upstream steps. In the context of RNA conformation and stability, magnesium cations, for example, are critical for proper folding of RNA and neutralize the negative charges on the backbone, allowing anionic groups to come into close proximity. However, under certain conditions, divalent metals are notoriously hazardous to the integrity of polynucleotides. The impact of pH, temperature, and metal ions on the transesterification reaction and the degradation of RNA has been thoroughly explored. Studies have shown that the chemical rate of SN2 attack by the nucleophilic oxygen on the adjacent phosphate group is accelerated by divalent metals and alkali conditions (Li and Breaker, Kinetics of RNA Degradation by Specific Base Catalysis of Transesterification Involving the 2′-Hydroxyl Group, J. Am. Chem. Soc. 121 (1999) 5364-5372). In the A-form helical structure of dsRNA, the phosphodiester groups are constrained and not well-positioned for strand scission compared to flexible, single-stranded regions that are more easily hydrolyzed. This highlights the importance of reinforcing RNA stability by optimizing mRNA construct design at the sequence level to stabilize the folded structure. While it may be difficult to fully remove contaminating metals from preparative processes, the use of additives can help stabilize polynucleotides and minimize the impact of magnesium and other divalent metal-dependent reactions.

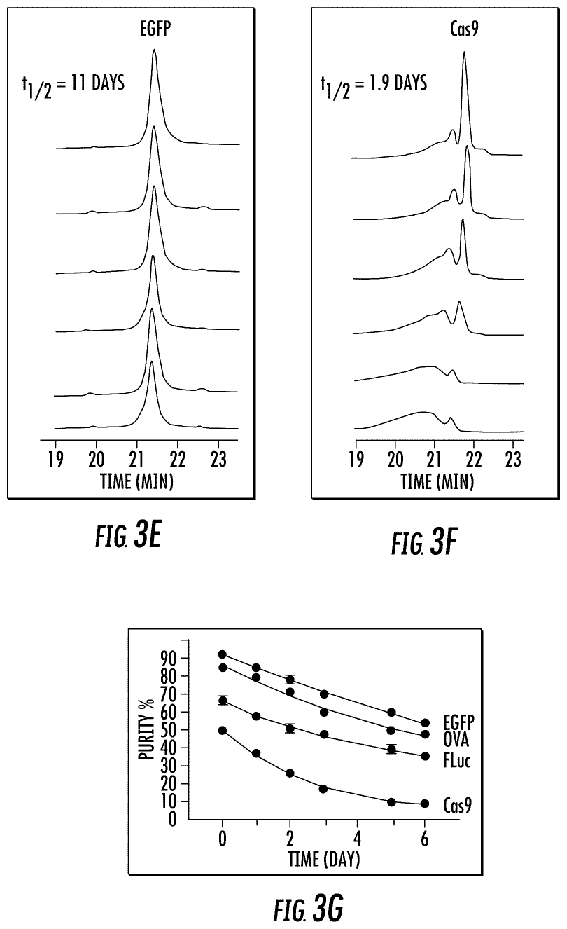

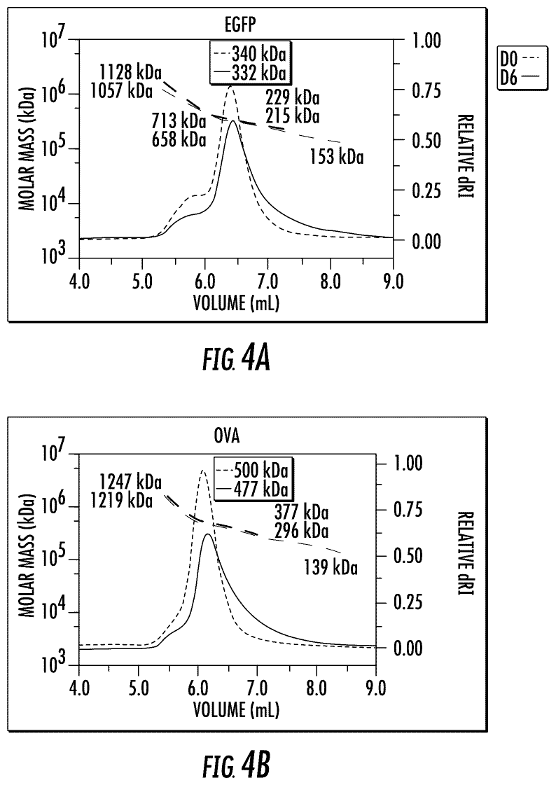

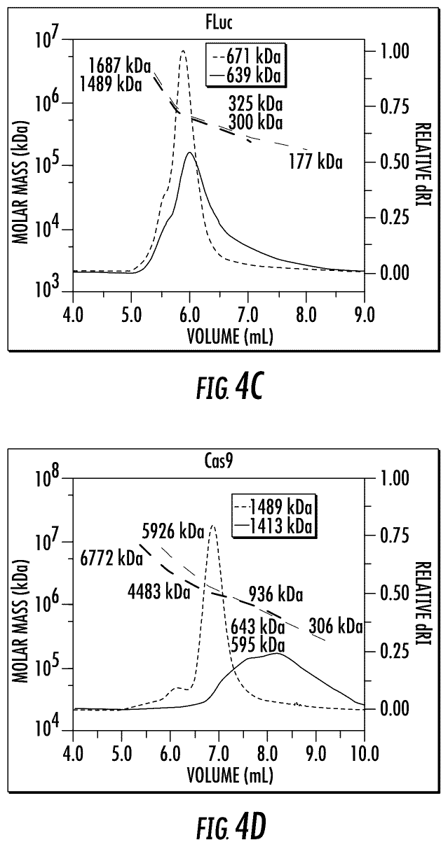

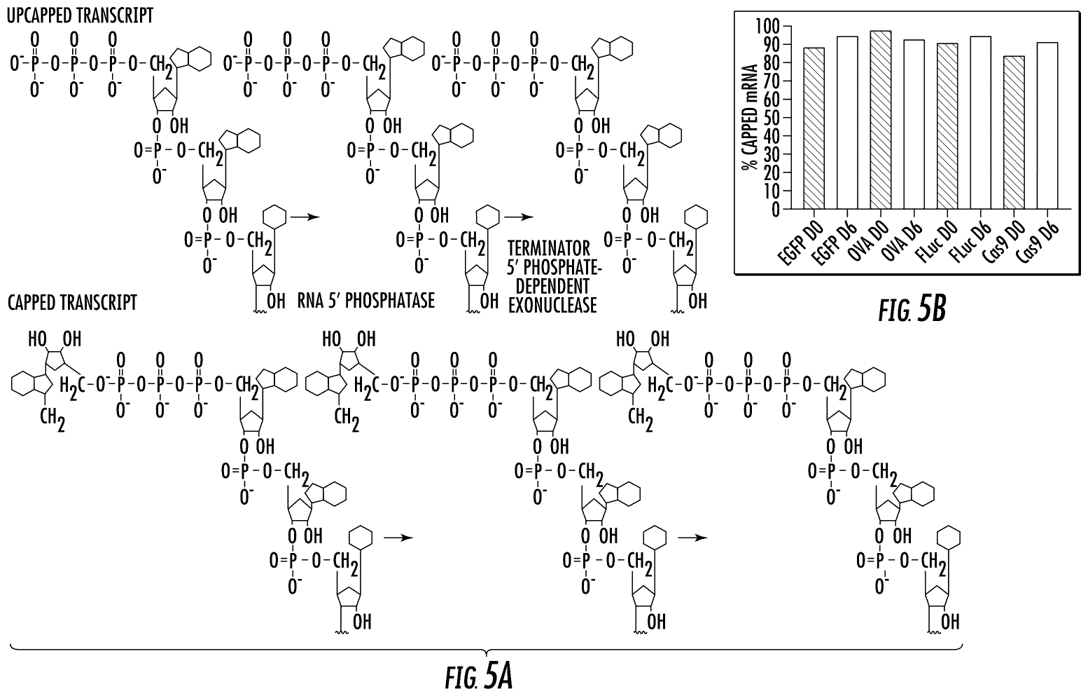

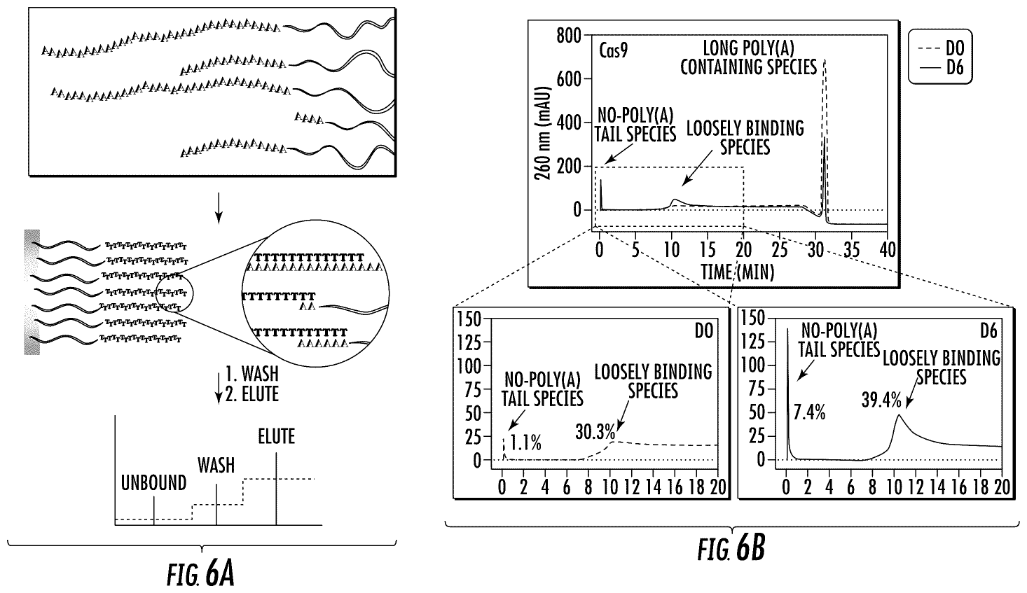

The current inventions outline an efficient strategy for the rapid assessment of quality attributes of RNA therapeutics using a series of stability-indicating assays. Using standard enzymatic treatments coupled with common chromatography tools appended with UV, RI, and MALS detectors, a series of thermally stressed transcripts were evaluated for their compositional purity (FIGS. 3A-3G), structural homogeneity and size distribution (FIGS. 4A-4D), and integrity of the 5′ cap and 3′ poly(A) tail (FIGS. 5A-5B and 6A-6B). The approach provides purity data on mRNA products consistent with those proposed by USP draft guidelines for mRNA vaccine quality while offering improvements that allow for the development of platform methods.

A summary of analytical methods used for the determination and characterization of mRNA quality attributes are provided in FIGS. 14 and 15.

The methodologies of the present inventions can be used across various stages of the drug development pipeline. For early-stage studies, these methods can help assess construct design, formulation conditions, and sample handling and technique. Later in the pipeline, these methods can be optimized for specific mRNAs for use in quality control space such as for release methods and lot-to-lot comparability studies. The assays shown here not only provide a roadmap for characterizing IVT mRNA but also for monitoring the integrity of specific components during the developmental process and when conducting stress studies. It is anticipated that as RNA therapeutics continue to grow, so too will the development of tools and methods to characterize drug products to ensure drug product quality, safety, and efficacy.

The inventions are further described by the following Examples, which do not limit the inventions in any manner and are applicable to all sections of the descriptions of the inventions and the aspects of the inventions. The order of performance of the below examples can be altered or combined as determined by the person of skill in the art in view of the teachings and data contained herein.

### Examples

### Thermal Stress and Sample Preparation

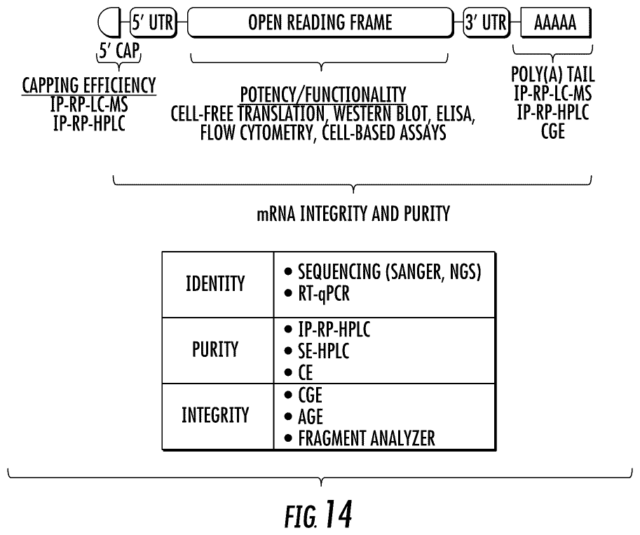

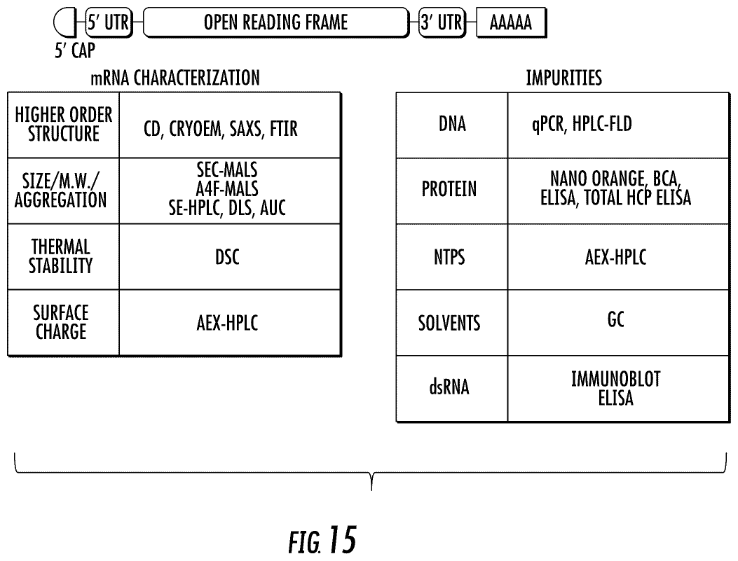

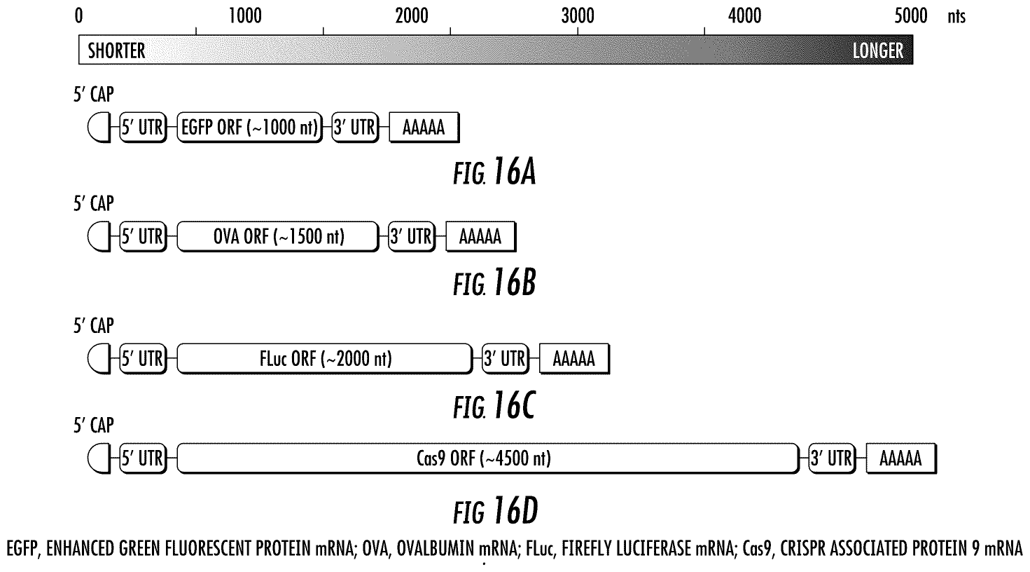

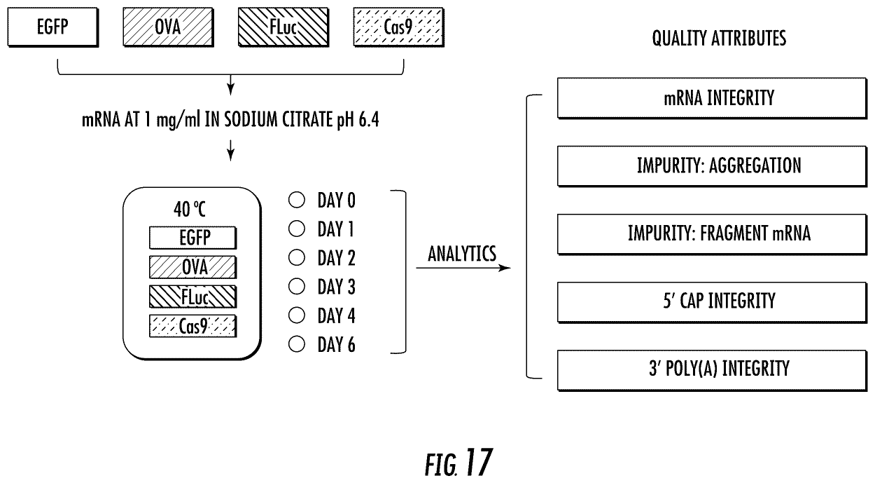

All mRNAs used in herein were purchased from TriLink Biotechnologies (San Diego, CA). Messenger RNA constructs used are schematically depicted in FIGS. 16A-16D. Messenger RNA samples with 5moU modification (EGFP, OVA, FLuc and Cas9 transcripts) were purchased from TriLink Biotechnologies (San Diego, CA) at a concentration of 1 mg/mL in 1 mM sodium citrate (pH 6.4). The mRNA samples were aliquoted into 100 μL and incubated at 40° C. for up to 6 days with time points taken at days 0, 1, 2, 3, 5, and 6. An overview of the process of thermal stress testing of the mRNA transcripts carried out and analysis of the quality attributes are schematically depicted in FIG. 17. Aliquots taken at each time point were immediately frozen and stored at −80° C. until analysis. For experiments in which EDTA was added to examine divalent metal contamination, sample preparation was conducted as described above but with 1 mM RNase-free EDTA (Thermo Fisher Scientific) added to the solution prior to thermal incubation.

Purity Analysis of in vitro transcription (IVT)-Produced mRNA by Ion-Pairing Reversed Phase (IPRP) Liquid Chromatography

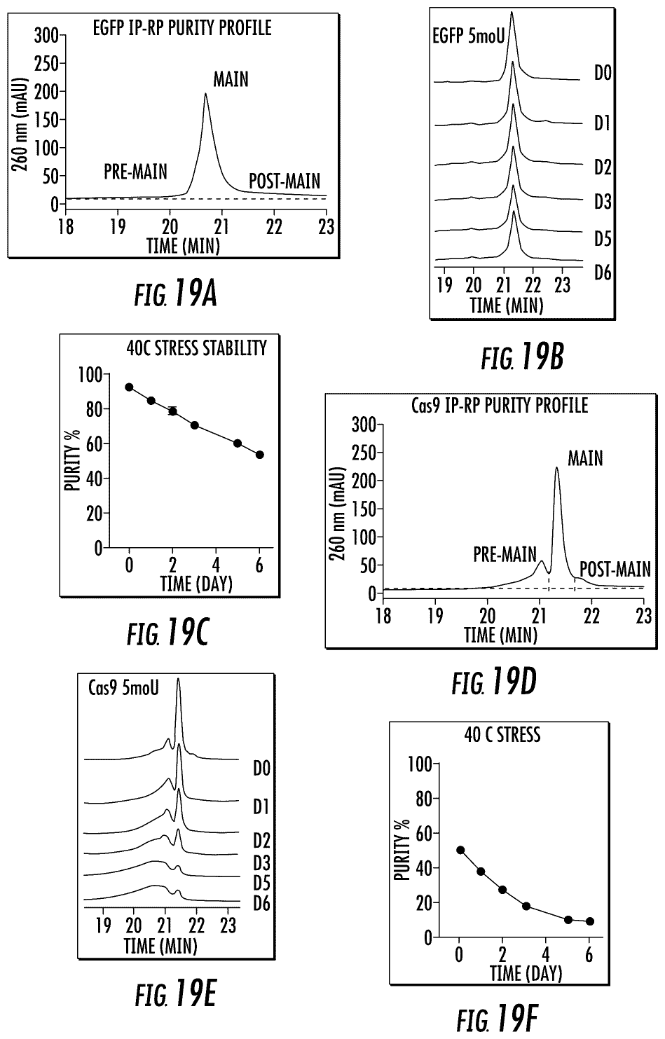

Purity of IVT-produced mRNA was analyzed using an adapted IPRP HPLC method on an Agilent 1290 Infinitely (Agilent Technologies) with a Thermo DNApac RP (4 μm, 2.1×100 mm) column heated to 75° C. Mobile phase A contained 100 mM triethylamine (TEA) in nuclease-free water and final pH adjusted to pH 8.5, and mobile phase B (MPB) contained 100 mM TEA and 40% (v/v) acetonitrile in nuclease-free water adjusted to pH 8.5. For all IPRP runs, 0.5 μg of each mRNA was injected onto a pre-equilibrated column, and samples were eluted using a linear gradient from 20%-40% MPB over 30 minutes at 0.2 mL/min. Data were recorded using a diode-array detector (Agilent Technologies) collecting at 260 nm. For half-life calculation, main peak area was plotted as a function of time, and data points were fitted to a single-phase decay model using GraphPad (Prism 9.4.1). Integrity profiles of mRNA transcripts EGFP and Cas9 are schematically depicted in FIGS. 19A to 19F.

Characterization of mRNA Size Distribution

Size distribution experiments were carried out on an Agilent 1260 HPLC interfaced with a Wyatt DAWN™ light scattering detector and Wyatt Optilab™ T-rEX refractive index detector. EGFP, OVA, and FLuc mRNA samples were injected neat onto a WTC050S5 500 Å, 7.8×300 mm, 5 μm (Wyatt Technology; Santa Barbara, CA) column, and CAS9 mRNA samples were injected neat onto a WTC100S5 1000 Å, 7.8×300 mm, 5 μm (Wyatt Technology; Santa Barbara, CA) column. Columns were pre-equilibrated with PBS, pH 7.4 supplemented with 1 mM EDTA at a flow rate of 0.5 mL/min. Molar masses were obtained from triplicate 10 μg injections of each mRNA sample using the refractive index detector as the concentration source (dn/dc_{RNA}=0.172 mL/g), where dn is the change in refractive index and dc is the change in concentration of the solute. For comparison, the theoretical molar mass for each mRNA was calculated from the manufacturer-provided transcript length using an average molecular weight of 330 Da per RNA nucleotide and 1,145 Da for the CleanCap moiety.

### 5′ Capping Analysis

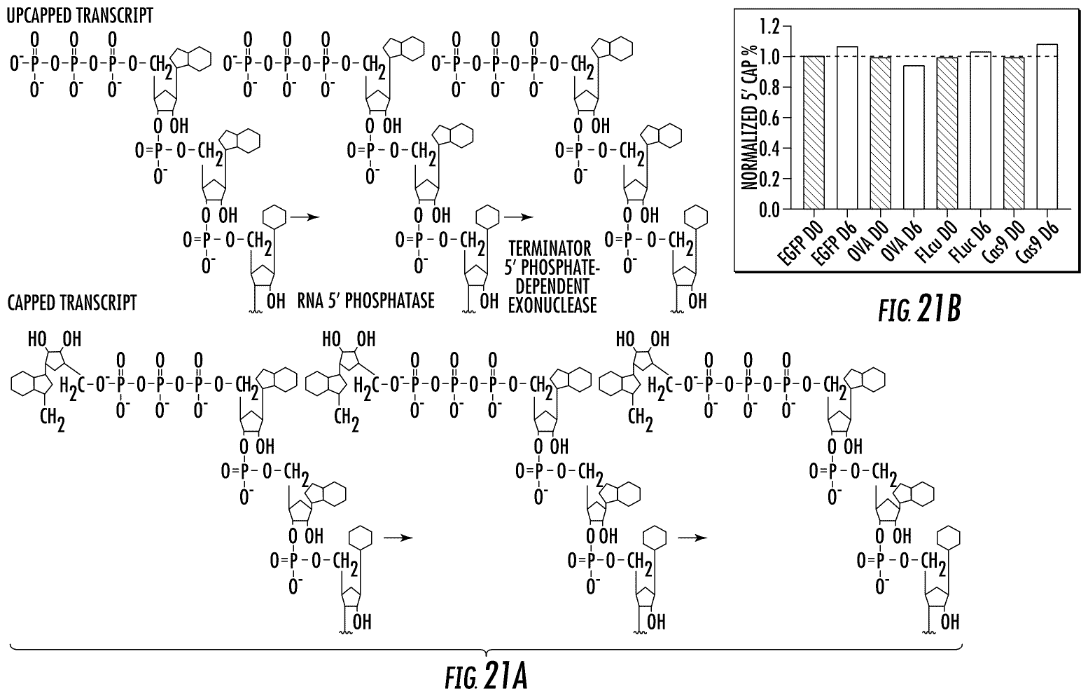

The enzymatic steps of 5′ capping efficiency analysis were carried out by sequential digestion using 5′ polyphosphatase (LGC Biosearch Technologies, cat #RP8092H) and terminator 5′ phosphate-dependent exonuclease (LGC Biosearch Technologies, cat #TER51020) as described in Sahin and Roberts (Size-exclusion chromatography with multi-angle light scattering for elucidating protein aggregation mechanisms., Methods Mol. Biol. (Clifton, NJ). 899 (2012) 403-23). Following the enzymatic digestion, only mRNA with 5′ cap remained intact while uncapped transcripts were readily degraded (FIGS. 5A and 21A). Intact transcripts and degraded fragments were resolved by size-exclusion chromatography (SEC) and the percentage of capped mRNA was calculated by comparing the remaining peak area to the peak area of the undigested control using the following equation:

$${5}^{′} Cap \% = \frac{main peak area of mRNA treated with enzymes}{main peak area of mRNA not treated with enzymes} \times 100$$

Analysis of 3′ Poly(A) Tail using Oligo-dT Affinity Capture

Poly(A) tail purity was evaluated by oligo-dT affinity capture on an Agilent 1290 HPLC system. Stressed samples were supplemented with 1 M nuclease-free NaCl (Thermo Fisher Scientific) before loading 10 μg of each mRNA sample onto a CIMac™ Oligo dT18 column (BIA Separations, Sartorius) pre-equilibrated with 50 mM Tris-HCl pH 7.0, 1 M NaCl, and 5 mM EDTA. After injection of each sample, the flow-through containing transcripts unable to bind the oligo-dT column were collected. The column was washed with 90 column volumes wash buffer (50 mM Tris-HCl pH 7.0, 100 mM NaCl, and 5 mM EDTA) to collect loosely bound transcripts before eluting mRNAs with intact poly(A) tails still bound to the column with 60 column volumes nuclease-free water (FIG. 6A). The column was regenerated with 100 mM NaOH.

Purity Analysis of Stressed mRNAs

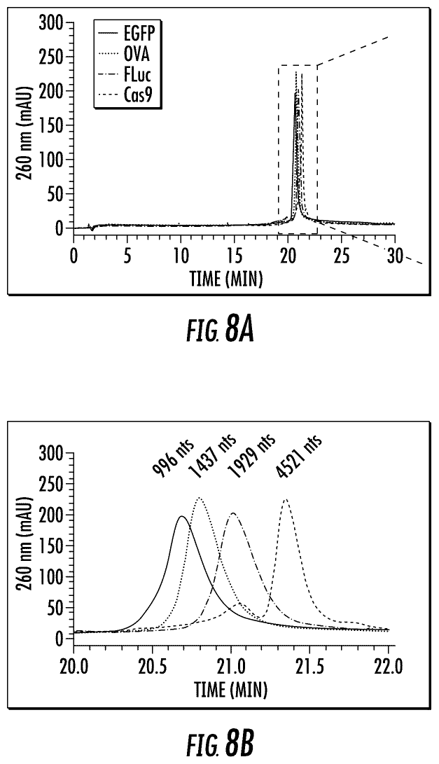

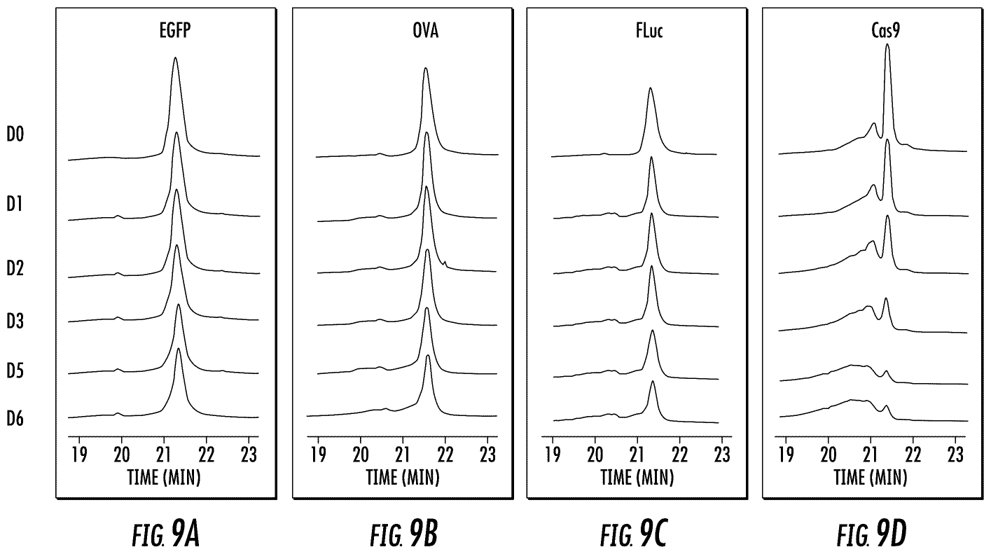

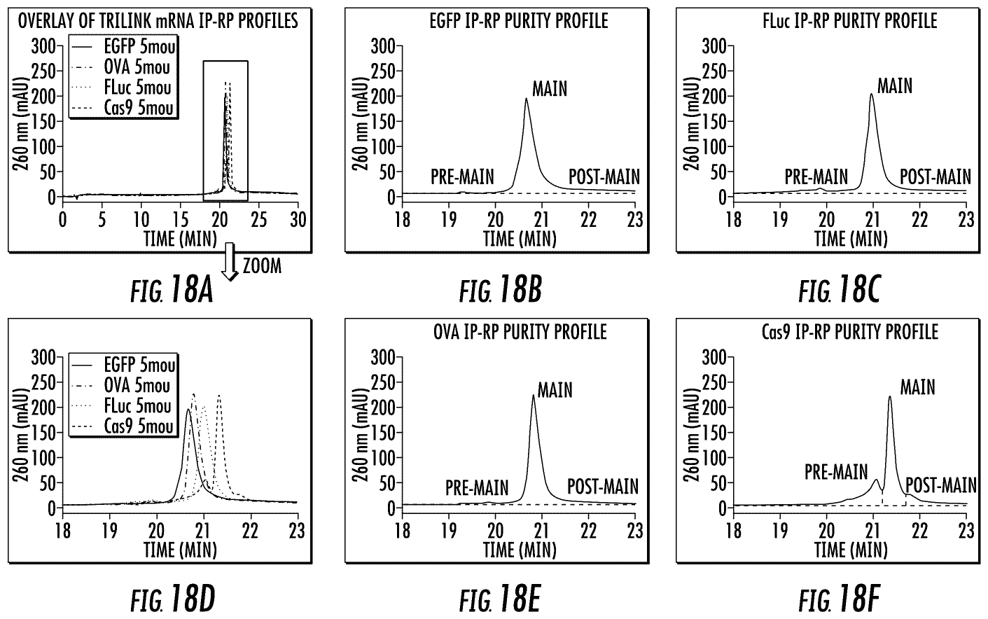

Total RNA purity of the stressed transcripts was evaluated by denaturing ion-paired reverse phase (IPRP) liquid chromatography. Initial analysis of the mRNAs at Day 0 revealed 3 populations in the chromatograms; 1) a large main peak expected to represent the correct full-length mRNA, 2) an early pre-main peak comprised of low molecular weight (LMW) truncated or partially degraded mRNAs, and 3) a post-main peak where longer impurities, termed “long-mers” or high molecular weight (HMW) impurities, eluted later owing to their elevated hydrophobicity provided by their extended length (FIGS. 3A to 3D, FIGS. 8A and 8B, and FIGS. 18A to 18F). The 4,521 nt Cas9 transcript (FIGS. 3D and 18F) exhibited considerably higher levels of pre-main and post-main impurities compared to the much shorter 996 nt EGFP transcript (FIGS. 3A and 18B) and 1,437 nt OVA transcript (FIGS. 3B and 18E). The main peak purity of the EGFP transcript reduced from about 95% to about 55% over the course of 6 days (FIGS. 3E, 9A, 19B, 19C and 3G), while the Cas9 main peak was almost completely diminished with only 20% of the initial main peak remaining at Day 6 (FIGS. 3F, 9D, 19E, 19F, and 3G). The calculated half-lives of the four transcripts are consistent with the previous observation that longer transcripts (FIGS. 16A to 16D) degraded faster (FIG. 3G, FIGS. 9A to 9D and Table 2).

Size Distribution of Stressed mRNAs

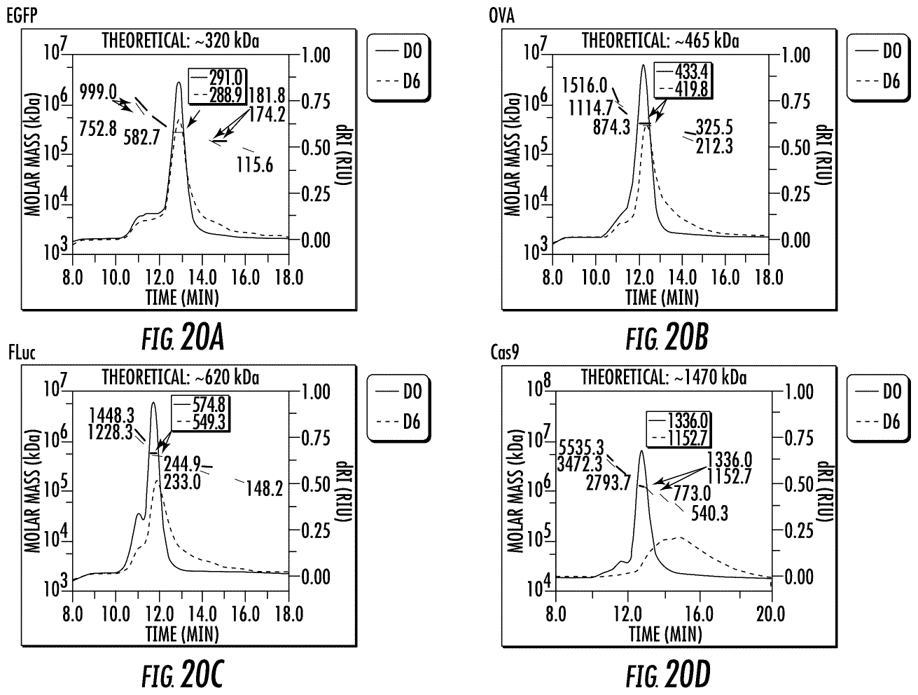

To gain more insights into the conformational purity and size distribution of the transcripts under non-denaturing conditions, size-exclusion chromatography was performed on the four mRNAs comparing initial and stressed samples. Interfacing SEC separation with multi angle light scattering (MALS) allowed determination of the molar mass of the eluting sample, providing a deeper characterization of the different populations in the sample and confirming that the transcript reflects the correct molecular weight of the designed sequence. The main population of all four transcripts was initially confirmed to be within the expected molecular weight ranges based on calculated theoretical masses (FIGS. 4A to 4D). The sensitivity of MALS detection allows calculation of molar masses of subpopulations present at low levels in a sample (FIGS. 4A to 4D, FIGS. 20A to 20D; FIG. 10, and Tables 3 to 6).

TABLE 3
Summary of molar mass determined using SEC-MALS
for HMW, main peak and LMW of EGFP at Day 0 and Day 6.
All data were shown in triplicates with average and standard deviation.
EGFP Molar Masses

| | | HMW | HMW | | LMW | LMW |
|---|---|---|---|---|---|---|
| | | 1 | 2 | Main | 1 | 2 |
| | Injection | (kDa) | (kDa) | (kDa) | (kDa) | (kDa) |
| D0 | 1 | 1148 | 686 | 338 | 198 | n.d. |
| | 2 | 1155 | 693 | 338 | 235 | n.d. |
| | 3 | 1293 | 759 | 344 | 255 | n.d. |
| | Average | 1199 | 713 | 340 | 229 | n.d. |
| | Standard | 82 | 40 | 3 | 29 | n.d. |
| | Deviation | | | | | |
| D6 | 1 | 955 | 626 | 330 | 210 | 139 |
| | 2 | 1150 | 687 | 333 | 228 | 181 |
| | 3 | 1066 | 661 | 332 | 208 | 139 |
| | Average | 1057 | 658 | 332 | 215 | 153 |
| | Standard | 98 | 31 | 2 | 11 | 24 |
| | Deviation | | | | | |

TABLE 4
Summary of molar mass determined using SEC-MALS for
HMW, main peak and LMW of OVA at Day 0 and Day 6.
All data were shown in triplicates with average and standard deviation.
OVA Molar Masses

| | | HMW 1 | HMW 2 | Main | LMW 1 |
|---|---|---|---|---|---|
| | Injection | (kDa) | (kDa) | (kDa) | (kDa) |
| D0 | 1 | 1204 | 500 | 335 | n.d. |
| | 2 | 1284 | 500 | 366 | n.d. |
| | 3 | 1267 | 499 | 370 | n.d. |
| | Average | 1252 | 500 | 357 | n.d. |
| | Standard | 42 | 0 | 19 | n.d. |
| | Deviation | | | | |
| D6 | 1 | 1181 | 474 | 292 | 160 |
| | 2 | 1248 | 479 | 296 | 108 |
| | 3 | 1229 | 479 | 300 | 149 |
| | Average | 1219 | 477 | 296 | 139 |
| | Standard | 35 | 3 | 4 | 27 |
| | Deviation | | | | |

TABLE 5
Summary of molar mass determined using SEC-MALS
for HMW, main peak and LMW of FLuc at Day 0 and Day 6.
All data were shown in triplicates with average and standard deviation.
Fluc Molar Masses

| | | HMW 1 | Main | LMW 1 | LMW 2 |
|---|---|---|---|---|---|
| | Injection | (kDa) | (kDa) | (kDa) | (kDa) |
| D0 | 1 | 1605 | 668 | 285 | n.d. |
| | 2 | 1634 | 673 | 394 | n.d. |
| | 3 | 1644 | 673 | 372 | n.d. |
| | Average | 1628 | 671 | 351 | n.d. |
| | Standard | 20 | 3 | 58 | n.d. |
| | Deviation | | | | |
| D6 | 1 | 1817 | 646 | 303 | 194 |
| | 2 | 1757 | 644 | 319 | 199 |
| | 3 | 1666 | 628 | 277 | 139 |
| | Average | 1747 | 639 | 300 | 177 |
| | Standard | 76 | 10 | 21 | 33 |
| | Deviation | | | | |

In addition to the main population, all four transcripts displayed oligomers that were consistent with dimeric or trimeric forms of the transcripts. Some higher molecular weight aggregates were also observed in the Cas9 mRNA sample. Following the 6-day thermal stress, EGFP, OVA, and FLuc transcripts began to display increased tailing as the mRNAs broke down and accumulated LMW species. The latter elution of mRNAs in the folded state reflects molecules with a generally smaller hydrodynamic radii, which is consistent with the observed breakdown of transcripts and generation of shortened oligomers, known as short-mers, detected by IPRP (FIGS. 3A to 3F). Degradation of the longer Cas9 transcript was relatively more pronounced and resulted in an entire population shift with little of the full-length population remaining after thermal stress. This data provides an understanding of the species present and serves as a proof of concept for the use of SEC coupled to MALS (SEC-MALS) for the analysis of mRNAs under native conditions.

TABLE 6
Summary of molar mass determined using SEC-MALS for
HMW, main peak and LMW of Cas9 without EDTA at Day 0 and Day 6.
All data were shown in triplicates with average and standard deviation.
Cas9 Molar Masses

| | | HMW | HMW | | LMW | LMW | LMW |
|---|---|---|---|---|---|---|---|
| | | 1 | 2 | Main | 1 | 2 | 2 |
| | Injection | (kDa) | (kDa) | (kDa) | (kDa) | (kDa) | (kDa) |
| D0 | 1 | 6240 | 2958 | 1495 | 610 | n.d. | n.d. |
| | 2 | 6482 | 2985 | 1496 | 605 | n.d. | n.d. |
| | 3 | 7593 | 3177 | 1503 | 714 | n.d. | n.d. |
| | Average | 6772 | 3040 | 1498 | 643 | n.d. | n.d. |
| | Standard | 722 | 119 | 4 | 62 | n.d. | n.d. |
| | Deviation | | | | | | |
| D6 | 1 | n.d. | 6907 | 1396 | 930 | 599 | 310 |
| | 2 | n.d. | 8532 | 1504 | 969 | 607 | 323 |
| | 3 | n.d. | 2340 | 1338 | 908 | 577 | 285 |
| | Average | n.d. | 5926 | 1413 | 936 | 595 | 306 |
| | Standard | n.d. | 3210 | 84 | 31 | 16 | 19 |
| | Deviation | | | | | | |

5′ Cap Analysis of Stressed mRNAs

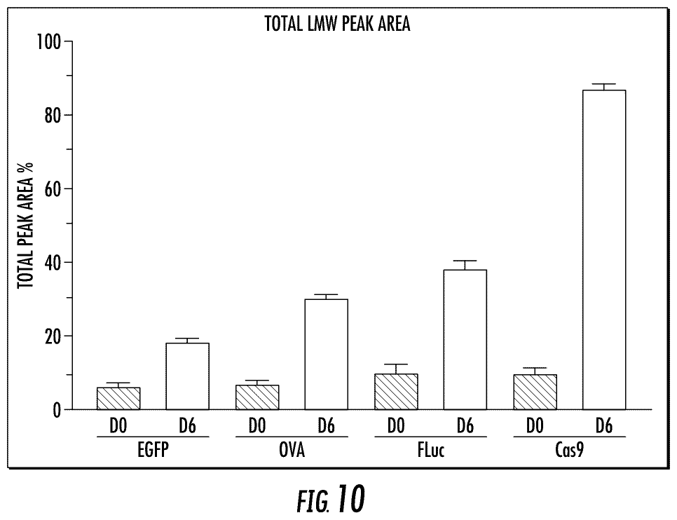

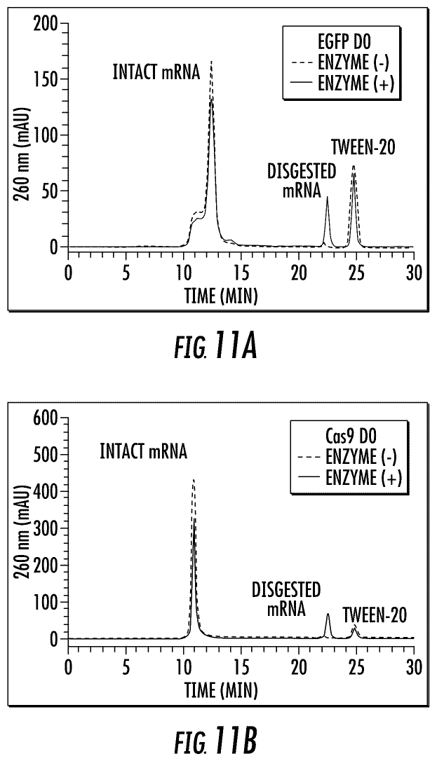

Enzymatic protocols coupled with liquid chromatography are effective approaches to evaluate the status of the 5′ cap of IVT mRNA. To determine if degrading transcripts retained their 5′ cap structure, an enzymatic approach was employed. The transcripts are treated with enzymes in sequence capable of degrading mRNA from the 5′ end. The transcripts retaining their 5′ cap are protected from degradation while those that no longer retain an intact cap structure are readily broken down (FIGS. 5A and 21A). Comparison of intact mRNA populations between enzyme-treated and untreated samples enables quantification of capped mRNAs. When performed for the four commercial transcripts tested here, the 5′ cap analysis revealed that the status of the cap structures of all four transcripts was relatively unchanged after incubation (FIGS. 5B and 21B, FIGS. 11A to 11B, FIGS. 22A to 22H; and Table 7), indicating that degradation of the mRNAs did not occur from the 5′ end.

These findings highlight the stability of the 5′ cap analog used here and underscore the importance of 5′ capping towards the preservation of the coding sequence against 5′ exonucleases.

TABLE 7
Summary of 5' Cap percentage for each mRNA transcript
at Day 0 and Day 6.

| mRNA | D0 5′ Cap % | D6 5′ Cap % |
|---|---|---|
| EGFP | 89 | 95 |
| OVA | 98 | 93 |
| FLuc | 92 | 95 |
| Cas9 | 85 | 92 |

3′ Poly(A) Tail Analysis of Stressed mRNAs

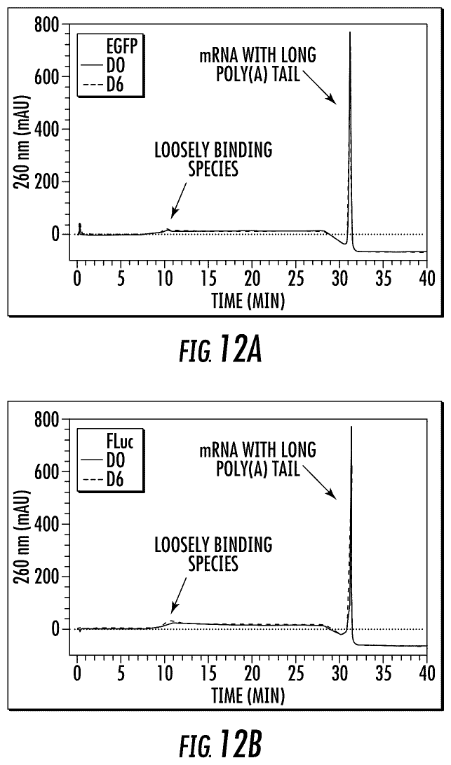

Given that the 5′ end of the transcripts remained intact, it was investigated whether the breakdown was occurring from the 3′ end. An oligo-dT affinity capture was used as a proxy for poly(A) integrity by probing a transcript's ability to bind to an oligo-dT stationary phase in a poly(A) tail-dependent manner. Transcripts with fully intact poly(A) tails will bind the column more tightly and require more elution buffer to disrupt the poly(A):oligo(dT) interaction, whereas transcripts lacking a poly(A) tail or whose tails are too short to interact with the column will be collected in the flow-through fraction (FIG. 6A). Meanwhile, mRNAs with shortened or partially degraded poly(A) tails are loosely bound to the oligo-dT stationary phase relative to those with intact poly(A) tails, and thus come off in the wash step. Comparison of the Cas9 oligo-dT capture and elution at initial and incubated time points exhibited a significant loss of long poly(A)-containing species, indicated by the reduction of the main peak comprised of mRNAs with long, intact poly(A) tails (FIG. 6B). Closer examination of the profiles revealed that the intact poly(A) tails had degraded over the course of the stress study, evidenced by the about 7-fold and about 1.8-fold increases in the unbound and wash populations, respectively (FIG. 6B and Tables 8A and 8B). FIGS. 12A and 12B depicts representative chromatograms for oligo-(dT) affinity capture for EGFP and FLuc mRNA, respectively. Individual profiles for each transcript showing different populations within each sample with different lengths of EGFP (FIG. 12A) and FLuc (FIG. 12B) poly(A) tails.

Table 8A and 8B. Quantified results for poly(A) purity assessment:

TABLE 8A
Quantified peak area for Cas9 mRNA flowthrough,
wash, and elution fractions for D0-D6 samples.

| | Days at 40° C. | Flow-through (%) | Wash (%) | Elution (%) |
|---|---|---|---|---|
| Cas9 | D0 | 1.1 | 30.3 | 68.6 |
| | D1 | 2.1 | 36.5 | 61.4 |
| | D2 | 3.5 | 40.4 | 56.0 |
| | D3 | 4.6 | 47.6 | 47.8 |
| | D5 | 7.6 | 52.9 | 39.5 |
| | D6 | 7.4 | 53.1 | 39.4 |

TABLE 8B
Quantified peak area for EGFP, OVA, and FLuc mRNAs
flowthrough, wash, and elution fractions for D0 and D6 samples.

| | Days at 40° C. | Flow-through (%) | Wash (%) | Elution (%) |
|---|---|---|---|---|
| EGFP | D0 | 0.6 | 6.7 | 92.6 |
| | D6 | 3.4 | 13.4 | 83.1 |
| OVA | D0 | 0.0 | 11.2 | 88.8 |
| | D6 | 1.2 | 17.3 | 81.5 |
| FLuc | D0 | 0.0 | 24.7 | 75.3 |
| | D6 | 0.1 | 34.1 | 65.8 |

Taken together, these experiments demonstrate that degradation occurs more readily at the 3′ end of the mRNA than the 5′ end, and the cap structure sufficiently protects the 5′ end of the transcript.

Inhibition of mRNA Degradation In Vitro

TABLE 9
A summary of degradation rate for different mRNAs is shown.

| | Half-life* | mRNA length | |
|---|---|---|---|
| mRNA | (Day) | (nt) | R² |
| EGFP (5moU) | 10.95 | 996 | 0.994 |
| OVA (5moU) | 5.13 | 1437 | 0.978 |
| FLUC (5moU) | 3.29 | 1929 | 0.976 |
| CAS9 (5moU) | 1.93 | 4521 | 0.994 |

*At 40° C., fitted using one-phase decay.

Based on results above, denaturing IPRP is proven to be stability indicating method to monitor degradation of mRNA.

Half-life of mRNA may be dependent on mRNA length. Longer mRNA is more prone to degradation.

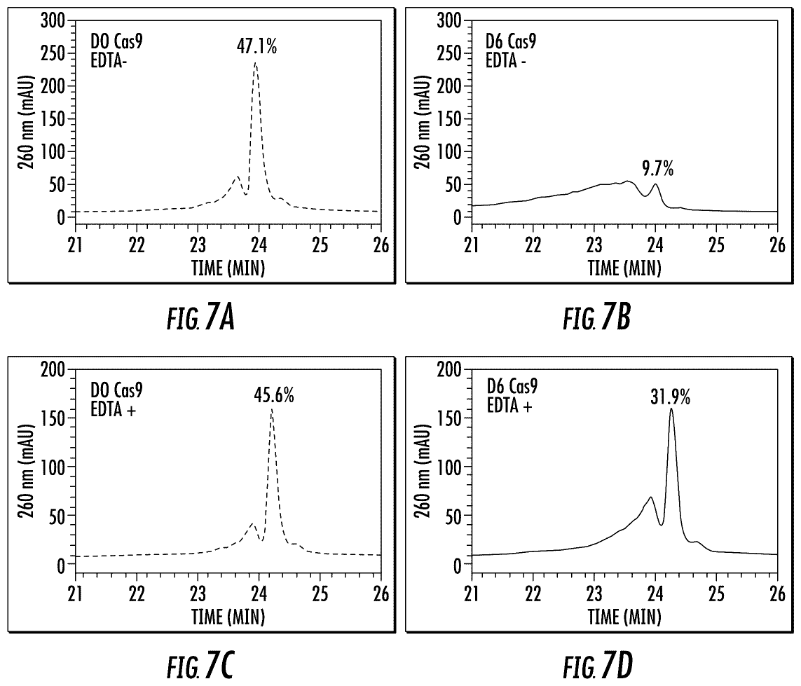

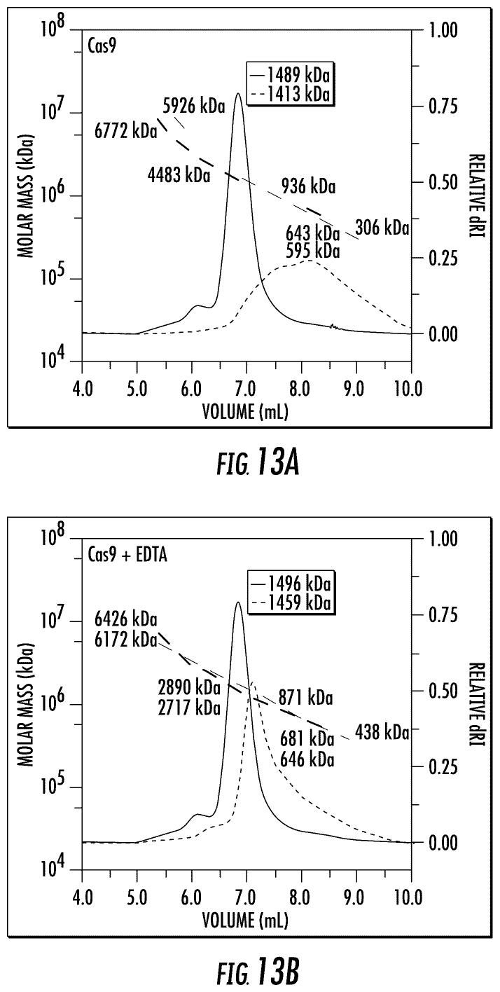

Despite the important role of metals in RNA folding and as cofactors in RNA-mediated reactions, divalent metals also accelerate RNA-degradation. It is possible that mRNA samples may contain trace levels of divalent metal contamination, which may have facilitated fragmentation of the mRNA transcript at elevated temperatures. If so, the addition of small amounts of EDTA would prevent RNA degradation and bolster the stability of IVT mRNAs. To this end, the 6-day stress experiment was repeated in the presence and absence of EDTA during incubation. The Cas9 mRNA was used as a model transcript for this experiment since it displayed the lowest integrity over the 6-day stress experiment (FIGS. 3A to 3D, FIGS. 4A to 4D and FIGS. 9A to 9D) and the impact of EDTA would be most evident. After 6 days, the purity of Cas9 mRNA was evaluated by IPRP and revealed that that the Cas9 transcript incubated without EDTA decreased by about 4.8-fold (FIGS. 7A and 7B). However, the main peak of the transcript that was incubated with EDTA was reduced by about 1.4-fold of its starting amount (FIGS. 7C and 7D). Molar mass analysis by SEC-MALS confirmed that the main peak of the sample supplemented with EDTA corresponded to the correct mass of full length Cas9 mRNA (FIGS. 13A and 13B; and Tables 8 and 10). These data indicate that mRNA breakdown in these stressed samples could be mediated by divalent metal contamination and is inhibited by the addition of EDTA to the buffer, suggesting that trace divalent metal contaminants are being chelated and thereby limiting hydrolysis. P_{GP}-32,T2

TABLE 10
Summary of molar mass determined using SEC-MALS
for HMW, main peak and LMW of Cas9 with EDTA at Day 0 and Day 6. All data were shown in
triplicates with average and standard deviation.
Cas9 + EDTA Molar Masses

| | | HMW 1 | HMW 2 | Main | LMW 1 | LMW 2 | LMW 2 |
|---|---|---|---|---|---|---|---|
| | Injection | (kDa) | (kDa) | (kDa) | (kDa) | (kDa) | (kDa) |
| D0 | 1 | 6102 | 2941 | 1497 | 664 | n.d. | n.d. |
| | 2 | 6517 | 3092 | 1480 | 632 | n.d. | n.d. |
| | 3 | 7421 | 3158 | 1510 | 748 | n.d. | n.d. |
| | Average | 6680 | 3064 | 1496 | 681 | n.d. | n.d. |
| | Standard | 674 | 111 | 15 | 60 | n.d. | n.d. |
| | Deviation | | | | | | |
| D6 | 1 | 4288 | 2554 | 1450 | 863 | 642 | 439 |
| | 2 | 5690 | 2684 | 1457 | 866 | 635 | 432 |
| | 3 | 8537 | 2912 | 1469 | 884 | 660 | 443 |
| | Average | 6172 | 2717 | 1459 | 871 | 646 | 438 |
| | Standard | 2165 | 181 | 10 | 11 | 13 | 6 |
| | Deviation | | | | | | |

mRNA Biophysical Characterization:
Uridine Base Modifications of In Vitro Transcribed mRNA Constructs of Varying Length and Temperature Mediated Unfolding/Refolding of High Molecular Weight/Aggregate Species

Commercial in vitro transcribed mRNA constructs of varying length and uridine base modifications were analyzed by SEC-MALS. In brief, samples were thawed from storage at −80° C., and 10 μg of each (12 constructs in total) were injected in triplicate onto the specified column hooked up to an SEC-MALS system consisting of an Agilent 1260 with DAD (UV) detector, Wyatt DAWN (18-angle multi-angle light scattering, MALS) detector, and Wyatt Optilab (RI) detector. EGFP and OVA constructs were injected onto a 500 Å, 5 μm, 7.8×300 mm (Wyatt WT-050S5) column, and Fluc and Cas9 were injected onto a 1000 Å, 5 μm, 7.8×300 mm (Wyatt WT-100S5) column equilibrated in 1× PBS, pH 7.4 at a flow rate of 0.5 mL/min. Molar masses and radius of gyration (Rg) were calculated using ASTRA software with a refractive index increment of 0.172 mL/g for mRNA (using the RI detector as the concentration source) and a Zimm model with a fit degree of 1. The delay volumes (i.e., detector alignment), band broadening between the UV, LS, and RI detectors, and LS detector normalization were determined using bovine serum albumin (BSA) standard.

Initial SEC-MALS data collection (panels A-C for all constructs) shows varying levels of high molecular weight/aggregate species, more so with the N1U constructs. By SEC-MALS these higher order species were found to be larger in mass and size (radius of gyration, Rg) than the monomeric main peak and reflected sizes that would align with dimer and larger oligomeric species.

Treating samples with a temperature mediated unfolding/refolding step (panels D-F for all constructs) resulted in disruption of the larger aggregates, with little to no change seen with the main, monomeric species molar mass or size (Rg). In brief, samples were thawed from storage at −80° C., heated to 65° C. at 2.5° C./min and held for 5 min, then brought down to 15° C. at 2.5° C./min. SEC-MALS data collection and analysis were performed.

This suggests N1U mRNA constructs have a higher propensity for HMW aggregate formation, and these aggregates are reversible and can be broken following a temperature mediated unfolding/refolding step without greatly perturbing the main monomeric species molar mass and size.

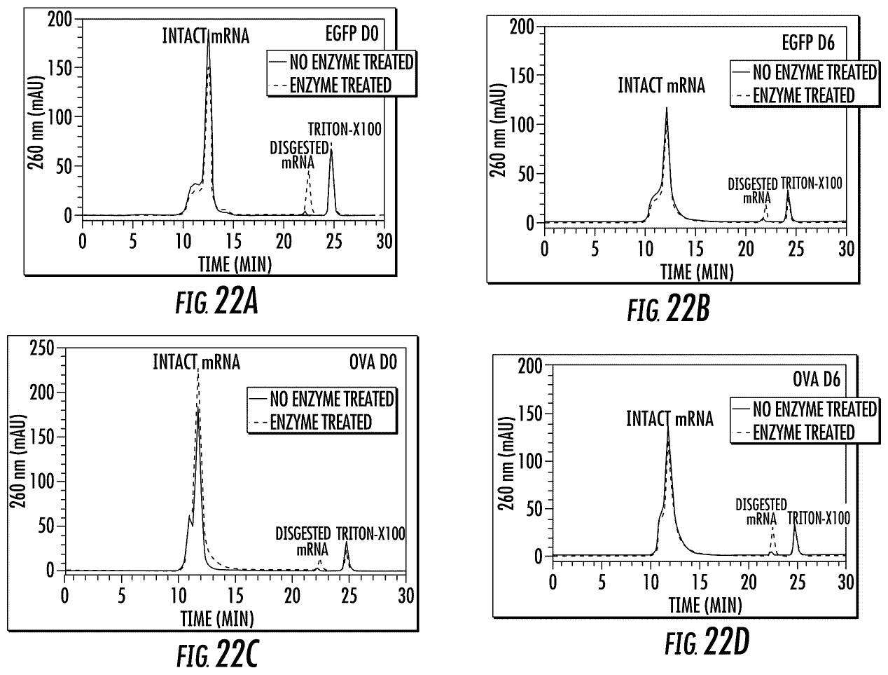

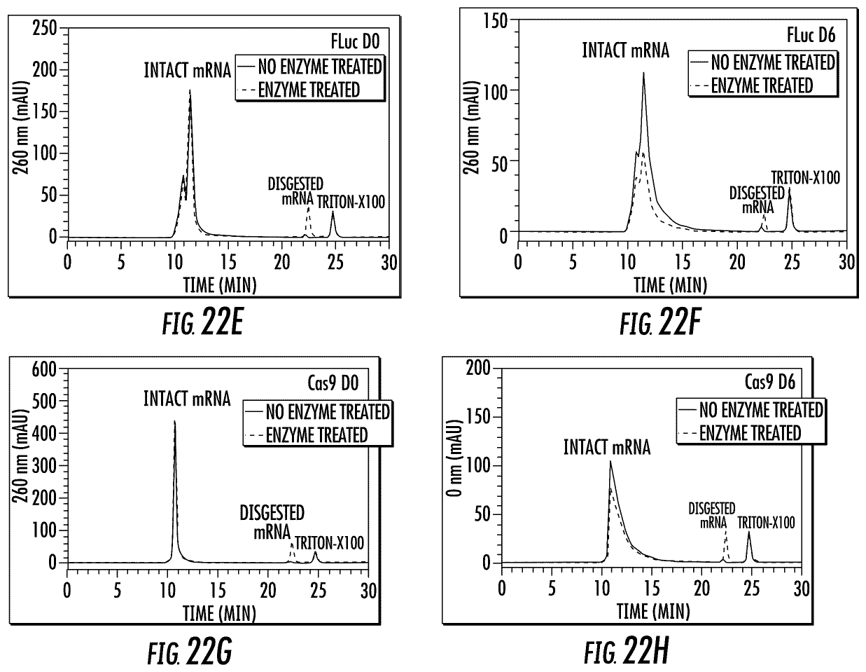

FIGS. 22A to 22H are graphs showing 5′ capping analysis SEC chromatograms for all four mRNA samples in the following order: EGFP, OVA, FLuc and Cas9 D0 (FIGS. 22A, 22C, 22E, and 22G) or D6 (FIGS. 22B, 22D, 22F, and 22H) samples. Samples were resolved by SEC, and percent (%) of capped mRNA is back calculated from the difference in peak area percentage between the treated and untreated samples. Triton X-100 peak was from the enzyme buffer used in the digestion (RNA 5′ polyphosphatase).

Comparison of intact mRNA populations between enzyme-treated and untreated samples enables quantification of capped mRNAs (FIGS. 22A to 22H. When performed for the four commercial transcripts tested here, the 5′ cap analysis revealed that the status of the cap structures of all four transcripts was relatively unchanged after incubation (also see FIGS. 5B and 21B, FIGS. 11A to 11B, and Table 7), indicating that degradation of the mRNAs did not occur from the 5′ end.

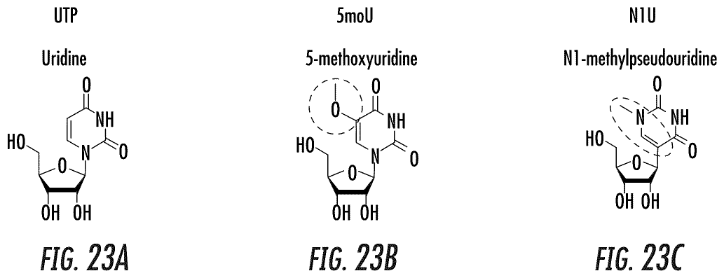

FIGS. 23A-23C: Uridine bases of in vitro transcribed mRNA constructs EGFP, OVA, FLUC, and CAS9 of varying length, as shown in Table 11, are modified as UTR (FIG. 23A), 5moU (FIG. 23B) and N1U (FIG. 23C) and for analysis by SEC-MALS.

TABLE 11
EGFP, OVA, FLUC, and CAS9 of varying lengths.

| Commercial | | Expected Molar |
|---|---|---|
| mRNA Construct | Length (nucleotides) | Mass (kDa) |
| EGFP | 996 | 330 |
| OVA | 1438 | 476 |
| Fluc | 1929 | 638 |
| Cas9 | 4521 | 1493 |

EGFP, OVA, FLUC, and CAS9 - all UTP bases were substituted with 5moU or N1U.

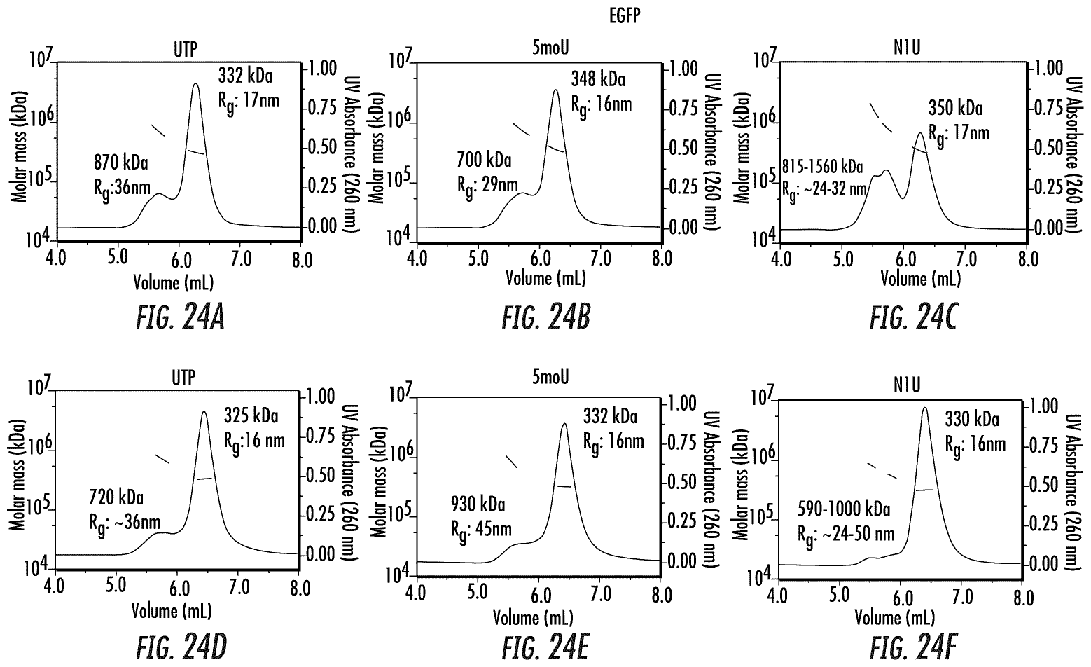

FIGS. 24A to 24F: FIGS. 24A, 24B and 24C depicting initial SEC-MALS data collection for EGFP constructs with uridine modified as UTR, 5moU and N1U, respectively. Data showing varying levels of high molecular weight/aggregate species. FIGS. 24D, 24E and 24F depicting SEC-MALS data for EGFP constructs with uridine modified as UTR, 5moU and N1U, respectively, that have been treated with a temperature mediated unfolding/refolding step. Data showing resulted disruption of the larger aggregates.

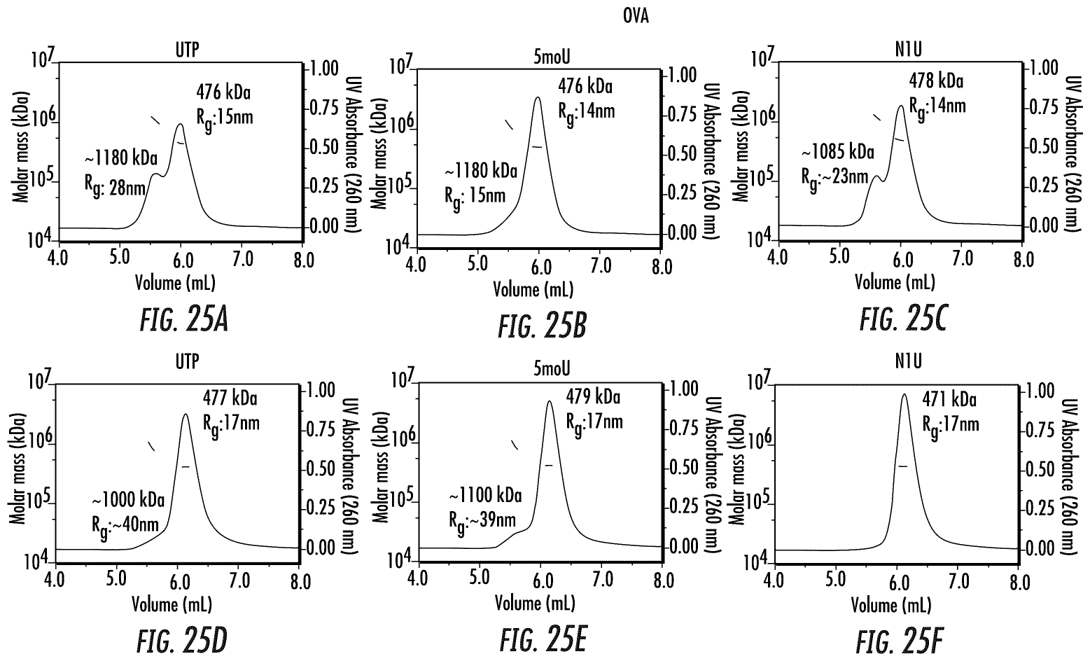

FIGS. 25A to 25F: FIGS. 25A, 25B and 25C depicting initial SEC-MALS data collection for OVA constructs with uridine modified as UTR, 5moU and N1U, respectively. Data showing varying levels of high molecular weight/aggregate species. FIGS. 25D, 25E and 25F depicting SEC-MALS data for OVA constructs with uridine modified as UTR, 5moU and N1U, respectively, that have been treated with a temperature mediated unfolding/refolding step. Data showing resulted disruption of the larger aggregates.

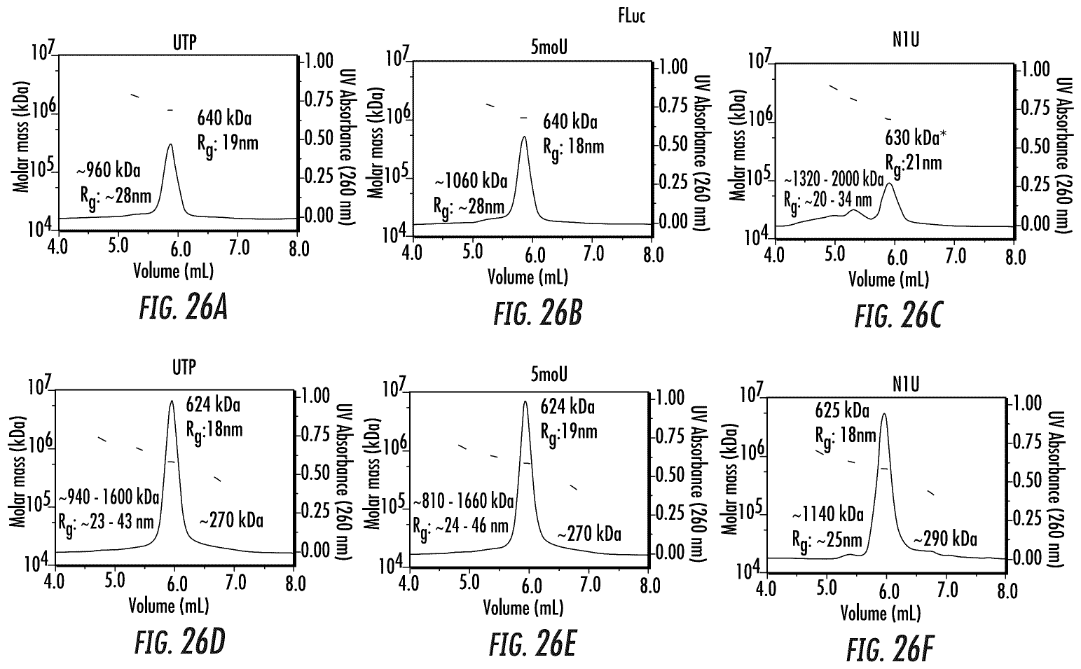

FIGS. 26A to 26F: FIGS. 26A, 26B and 26C depicting initial SEC-MALS data collection for FLuc constructs with uridine modified as UTR, 5moU and N1U, respectively. Data showing varying levels of high molecular weight/aggregate species. FIGS. 26D, 26E and 26F depicting SEC-MALS data for FLuc constructs with uridine modified as UTR, 5moU and N1U, respectively, that have been treated with a temperature mediated unfolding/refolding step. Data showing resulted disruption of the larger aggregates.

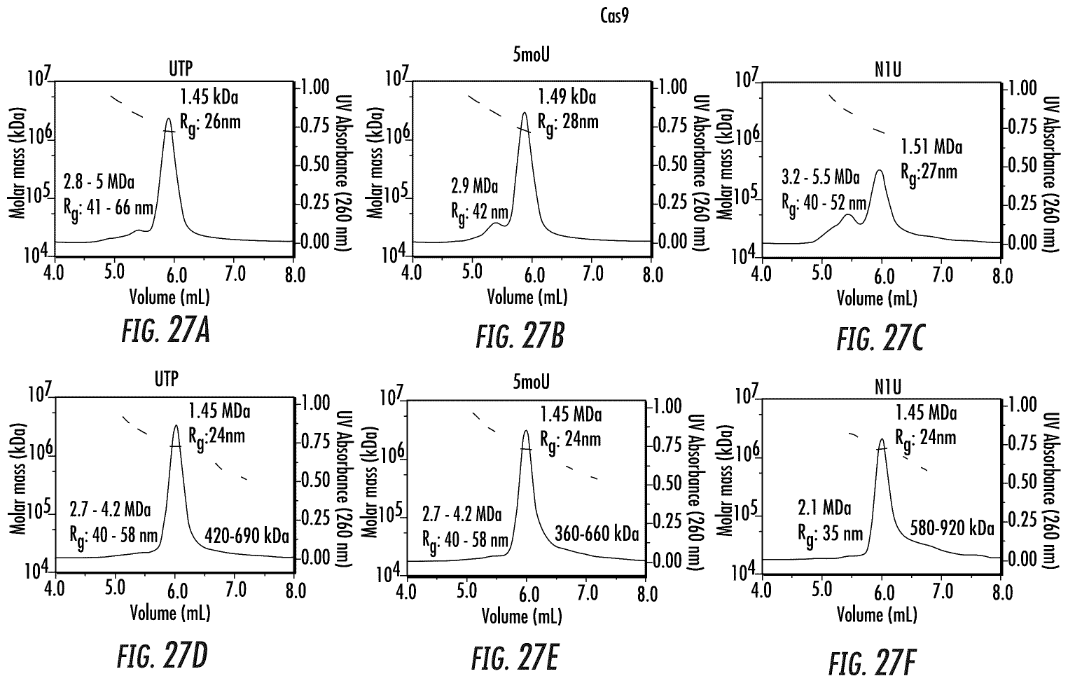

FIGS. 27A to 27F: FIGS. 27A, 27B and 27C depicting initial SEC-MALS data collection for Cas9 constructs with uridine modified as UTR, 5moU and N1U, respectively. Data showing varying levels of high molecular weight/aggregate species. FIGS. 27D, 27E and 27F depicting SEC-MALS data for Cas9 constructs with uridine modified as UTR, 5moU and N1U, respectively, that have been treated with a temperature mediated unfolding/refolding step. Data showing resulted disruption of the larger aggregates.

It is to be understood that the description, specific examples and data are given by way of illustration and are not intended to limit the present inventions. Various changes and modifications within the present inventions, including combining features in whole or in part, will become apparent to the skilled artisan from the discussion, disclosure and data contained herein, and thus are considered part of the inventions.

What is claimed is:

1. A method of evaluating or analyzing critical quality attributes (CQAs) of messenger ribonucleic acid (mRNA) molecules in a sample, wherein the method comprises the steps of:
(a) determining the purity of the mRNA contained in the sample using ion-paired reverse-phase (IPRP) liquid chromatography;
(b) determining the size distribution of the mRNA contained in the sample using size exclusion chromatography (SEC) for separation of different size molecules, and multi-angle light scattering (MALS) detector to determine the molar mass of the SEC eluted sample, thereby identifying species size distribution in the sample; and
(c) comparing the purity of the mRNA from step (a) and the SEC-MALS species size distribution from step (b) to intact mRNA, thereby evaluating or analyzing the CQAs of the mRNA molecules present in the sample.

2. The method according to claim 1, wherein an ultraviolet light (UV) detector or a refractive index (RI) detector can be used at step (b).

3. The method according to claim 1, wherein the mRNA is further evaluated for 5′ cap integrity by enzymatic digestion of the sample in combination with the SEC.

4. The method according to claim 1, wherein the mRNA is further evaluated for 3′ poly(A) integrity by oligo(dT) affinity capture.

5. The method according to claim 1, wherein the mRNA is analysed for size distribution, wherein the size distribution is determined by SEC-MALS.

6. The method according to claim 1, wherein the CQAs are selected from the group consisting of mRNA purity; mRNA integrity including 5′ cap integrity and 3′ poly(A) integrity; and impurities including mRNA aggregates, mRNA fragments, DNA, peptide or polypeptide, NTPs, solvents, and dsRNA.

7. The method according to claim 1, wherein the migration time and size of peaks are compared, wherein the migration time is a relative migration time.

8. The method according to claim 1, wherein the sample of step (a) shows a reduction in polyadenylated (poly(A)) mRNA species.

9. A characterized mRNA sample obtained according to the method of claim 1.

10. A system of evaluating critical quality attributes (CQAs) of messenger ribonucleic acid (mRNA) molecules in a sample, wherein the system comprises:
(a) ion-paired reverse-phase (IPRP) liquid chromatography for determining the purity of the mRNA contained in the sample;
(b) size exclusion chromatography (SEC) for determining the size distribution of the mRNA contained in the sample for separation of different size molecules, and multi-angle light scattering (MALS) detector to determine the molar mass of the SEC eluted sample, thereby identifying species size distribution in the sample; and
(c) comparing the purity of the mRNA from step (a) and the SEC-MALS species size distribution from step (b) to intact mRNA, thereby evaluating the CQAs of the mRNA molecules present in the sample.

11. The system according to claim 10, wherein an ultraviolet light (UV) detector or a refractive index (RI) detector can be used at step (b).

12. The system according to claim 10, wherein the mRNA is further evaluated for 5′ cap integrity by enzymatic digestion of the sample in combination with the SEC.

13. The system according to claim 10, wherein the mRNA is further evaluated for 3′ poly(A) integrity by oligo(dT) affinity capture.

14. The system according to claim 10, wherein the mRNA is analysed for size distribution, wherein the size distribution is determined by SEC-MALS.

15. The system according to claim 10, wherein the CQAs are selected from the group consisting of mRNA purity; mRNA integrity including 5′ cap integrity and 3′ poly(A) integrity; and impurities including mRNA aggregates, mRNA fragments, DNA, peptide or polypeptide, NTPs, solvents, and dsRNA.

16. A method of minimizing the degradation of mRNA during heat exposure comprising adding EDTA to a mRNA preparation prior to heat exposure, wherein the degradation of mRNA is characterized by analyzing the mRNA molecules in a sample, wherein the method comprises the steps of:
(a) determining the purity of the mRNA contained in the sample using ion-paired reverse-phase (IPRP) liquid chromatography;
(b) determining the size distribution of the mRNA contained in the sample using size exclusion chromatography (SEC) for separation of different size molecules, and multi-angle light scattering (MALS) detector to determine the molar mass of the SEC eluted sample, thereby identifying species size distribution in the sample; and
(c) comparing the purity of the mRNA from step (a) and the SEC-MALS species size distribution from step (b) to intact mRNA, thereby analyzing the condition of the mRNA molecules present in the sample.

17. The method according to claim 16, wherein an ultraviolet light (UV) detector or a refractive index (RI) detector can be used at step (b).

18. The method according to claim 16, wherein the mRNA is further evaluated for 5′ cap integrity by enzymatic digestion of the sample in combination with the SEC.

19. The method according to claim 16, wherein the mRNA is further evaluated for 3′ poly(A) integrity by oligo(dT) affinity capture.

20. The method according to claim 16, wherein the mRNA is analyzed for size distribution, wherein the size distribution is determined by SEC-MALS.

21. The method according to claim 16, wherein the mRNA is exposed to a temperature of about 35° C. to about 65° C. for up to 10 days or to a temperature of about 40° C. for up to 6 days.

22. The method according to claim 16, wherein the concentration of mRNA is about 0.01 mg/mL to 100 mg/mL.

23. The method according to claim 16, wherein the mRNA is treated with about 0.01 mM to about 100 mM RNase-free EDTA.
